SEQUENCE LISTING (41 sequences)

SEQ ID NO: 1 - protein, 98 aa, synthetic construct
QVQLKESGGGLVQPGGSLKLSCAASGFTFSSYGMSWVRQTADKRLELVATTNNNGGVTYYPDSVKGRFTISRDNAKNTLYLQMSSLQSEDTAMYYCAR

SEQ ID NO: 2 - protein, 113 aa, synthetic construct
SDVLMTQTPLSLPVSLGDQASISCRSSQSIVHSNGNTYLEWYLQKPGQSPKLLIYKVSNRFSGVPDRFSGSGSGTDFTVKISRVEAEDLGVYYCFQGSHVPYTFGGGTKLEIK

SEQ ID NO: 3 - protein, 5 aa, synthetic construct
SYGMS

SEQ ID NO: 4 - protein, 16 aa, synthetic construct
TNNNGGVTYYPDSVKG

SEQ ID NO: 5 - protein, 16 aa, synthetic construct
RSSQSIVHSNGNTYLE

SEQ ID NO: 6 - protein, 7 aa, synthetic construct
KVSNRFS

SEQ ID NO: 7 - protein, 9 aa, synthetic construct
FQGSHVPYT

SEQ ID NO: 8 - protein, 263 aa, synthetic construct
MKLPVRLLVLMFWIPASSSQVQLKESGGGLVQPGGSLKLSCAASGFTFSSYGMSWVRQTADKRLELVATTNNNGGVTYYPDSVKGRFTISRDNAKNTLYLQMSSLQSEDTAMYYCARYGYYAMDYWGQGISVTVSSGGGGSGGGGSSGGGSDVLMTQTPLSLPVSLGDQASISCRSSQSIVHSNGNTYLEWYLQKPGQSPKLLIYKVSNRFSGVPDRFSGSGSGTDFTVKISRVEAEDLGVYYCFQGSHVPYTFGGGTKLEIK

SEQ ID NO: 9 - protein, 10 aa, synthetic construct
ESKYGPPCPP

SEQ ID NO: 10 - protein, 19 aa, synthetic construct
MKLPVRLLVLMFWIPASSS

SEQ ID NO: 11 - protein, 27 aa, synthetic construct
FWVLVVVGGVLACYSLLVTVAFIIFWV

SEQ ID NO: 12 - protein, 70 aa, synthetic construct
MGGKPRRKNPQEGLYNELQKDKMAEAYSEIGMKGERRRGKGHDGLYQGLSTATKDTYDALHMQALPPRGS

SEQ ID NO: 13 - protein, 41 aa, synthetic construct
RSKRSRLLHSDYMNMTPRRPGPTRKHYQPYAPPRDFAAYRS

SEQ ID NO: 14 - protein, 42 aa, synthetic construct
KRGRKKLLYIFKQPFMRPVQTTQEEDGCSCRFPEEEEGGCEL

SEQ ID NO: 15 - protein, 16 aa, synthetic construct
SSGGGGSGGGGSSGGG

SEQ ID NO: 16 - protein, 499 aa, synthetic construct
MKLPVRLLVLMFWIPASSSQVQLKESGGGLVQPGGSLKLSCAASGFTFSSYGMSWVRQTADKRLELVATTNNNGGVTYYPDSVKGRFTISRDNAKNTLYLQMSSLQSEDTAMYYCARYGYYAMDYWGQGISVTVSSGGGGSGGGGSSGGGSDVLMTQTPLSLPVSLGDQASISCRSSQSIVHSNGNTYLEWYLQKPGQSPKLLIYKVSNRFSGVPDRFSGSGSGTDFTVKISRVEAEDLGVYYCFQGSHVPYTFGGGTKLEIKESKYGPPCPPCPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGS

SEQ ID NO: 17 - DNA, 1499 bp, synthetic construct
  source 1..1499: mol_type = other DNA
atgaaactgcccgtgagactgctggtgctgatgttttggattcccgcttcctcgagccaggtgcagctgaaagagtccggcggaggactggtgcagcctggcggatctctgaagctgagctgtgctgccagcggcttcaccttcagcagctacggcatgagctgggtgcgacagaccgccgacaagagactggaactggtggctaccaccaacaacaacggcggcgtgacctactaccccgacagcgtgaagggcagattcaccatctccagagacaacgccaagaacaccctgtacctgcagatgagcagcctgcagagcgaggacaccgccatgtactactgcgccagatacggctactacgccatggattactggggccagggcatcagcgtgaccgtgtctagcggaggcggcggatctggcggagggggatctagtggcggaggctctgacgtgctgatgacccagacacctctgagcctgccagtgtccctgggcgaccaggccagcatcagctgtagaagcagccagagcatcgtgcacagcaacggcaacacctacctggaatggtatctgcagaagcccggccagagccccaagctgctgatctacaaggtgtccaacagattcagcggcgtgcccgacagattctccggcagcggctctggcaccgacttcaccgtgaagatctccagggtggaagccgaggacctgggcgtgtactactgttttcaaggcagccacgtgccctacaccttcggcggaggcaccaagctggaaatcaaggagagcaagtacggcccaccctgccctccatgtccattttgggtgctggtggtggtgggaggcgtgctggcctgttattctctgctggtgaccgtggccttcatcatcttttgggtgcgcagcaagcggagccggctgctgcactccgactacatgaacatgaccccaagacggcccggaccaacaaggaagcactaccagccttatgcaccaccccgcgattttgcagcataccggagcaagagaggcaggaagaagctgctgtatatcttcaagcagcccttcatgcggcccgtgcagaccacacaggaggaggacggctgctcctgtaggttccctgaagaggaggagggaggatgcgagctgagagtgaagttttctaggagcgccgatgcaccagcataccagcagggacagaatcagctgtataacgagctgaatctgggcaggcgcgaggagtacgacgtgctggataagaggagaggacgggaccccgagatgggaggcaagccaaggcgcaagaacccccaggagggcctgtacaatgagctgcagaaggacaagatggccgaggcctatagcgagatcggcatgaagggagagcggagaaggggcaagggacacgatggcctgtaccagggcctgtccaccgccacaaaggacacctatgatgccctgcacatgcaggccctgcctccaaggggcagcgg

SEQ ID NO: 18 - DNA, 789 bp, synthetic construct
  source 1..789: mol_type = other DNA
atgaaactgcccgtgagactgctggtgctgatgttttggattcccgcttcctcgagccaggtgcagctgaaagagtccggcggaggactggtgcagcctggcggatctctgaagctgagctgtgctgccagcggcttcaccttcagcagctacggcatgagctgggtgcgacagaccgccgacaagagactggaactggtggctaccaccaacaacaacggcggcgtgacctactaccccgacagcgtgaagggcagattcaccatctccagagacaacgccaagaacaccctgtacctgcagatgagcagcctgcagagcgaggacaccgccatgtactactgcgccagatacggctactacgccatggattactggggccagggcatcagcgtgaccgtgtctagcggaggcggcggatctggcggagggggatctagtggcggaggctctgacgtgctgatgacccagacacctctgagcctgccagtgtccctgggcgaccaggccagcatcagctgtagaagcagccagagcatcgtgcacagcaacggcaacacctacctggaatggtatctgcagaagcccggccagagccccaagctgctgatctacaaggtgtccaacagattcagcggcgtgcccgacagattctccggcagcggctctggcaccgacttcaccgtgaagatctccagggtggaagccgaggacctgggcgtgtactactgttttcaaggcagccacgtgccctacaccttcggcggaggcaccaagctggaaatcaag

SEQ ID NO: 19 - DNA, 10203 bp, synthetic construct
  source 1..10203: mol_type = other DNA
ccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattctgcagtcgacggtaccgcgggcccgggatccgccaccatgaaactgcccgtgagactgctggtgctgatgttttggattcccgcttcctcgagccaggtgcagctgaaagagtccggcggaggactggtgcagcctggcggatctctgaagctgagctgtgctgccagcggcttcaccttcagcagctacggcatgagctgggtgcgacagaccgccgacaagagactggaactggtggctaccaccaacaacaacggcggcgtgacctactaccccgacagcgtgaagggcagattcaccatctccagagacaacgccaagaacaccctgtacctgcagatgagcagcctgcagagcgaggacaccgccatgtactactgcgccagatacggctactacgccatggattactggggccagggcatcagcgtgaccgtgtctagcggaggcggcggatctggcggagggggatctagtggcggaggctctgacgtgctgatgacccagacacctctgagcctgccagtgtccctgggcgaccaggccagcatcagctgtagaagcagccagagcatcgtgcacagcaacggcaacacctacctggaatggtatctgcagaagcccggccagagccccaagctgctgatctacaaggtgtccaacagattcagcggcgtgcccgacagattctccggcagcggctctggcaccgacttcaccgtgaagatctccagggtggaagccgaggacctgggcgtgtactactgttttcaaggcagccacgtgccctacaccttcggcggaggcaccaagctggaaatcaaggagagcaagtacggcccaccctgccctccatgtccattttgggtgctggtggtggtgggaggcgtgctggcctgttattctctgctggtgaccgtggccttcatcatcttttgggtgcgcagcaagcggagccggctgctgcactccgactacatgaacatgaccccaagacggcccggaccaacaaggaagcactaccagccttatgcaccaccccgcgattttgcagcataccggagcaagagaggcaggaagaagctgctgtatatcttcaagcagcccttcatgcggcccgtgcagaccacacaggaggaggacggctgctcctgtaggttccctgaagaggaggagggaggatgcgagctgagagtgaagttttctaggagcgccgatgcaccagcataccagcagggacagaatcagctgtataacgagctgaatctgggcaggcgcgaggagtacgacgtgctggataagaggagaggacgggaccccgagatgggaggcaagccaaggcgcaagaacccccaggagggcctgtacaatgagctgcagaaggacaagatggccgaggcctatagcgagatcggcatgaagggagagcggagaaggggcaagggacacgatggcctgtaccagggcctgtccaccgccacaaaggacacctatgatgccctgcacatgcaggccctgcctccaaggggcagcggagccacaaacttctccctgctgaagcaggccggcgatgtggaggagaatcctggaccaatgccacctccaaggctgctgttctttctgctgtttctgaccccaatggaggtgcggccagaggagccactggtggtgaaggtggaggagggcgacaacgccgtgctgcagtgtctgaagggcacaagcgatggccctacccagcagctgacatggtctagagagagccctctgaagccattcctgaagctgtccctgggcctgccaggcttaggaatccatatgaggcccctggctatctggctgtttatcttcaacgtgagccagcagatgggaggcttctatctgtgccagccaggaccacctagcgagaaggcatggcagcctggatggaccgtgaacgtggagggcagcggagagctgtttcgctggaacgtgagcgacctgggaggcctgggatgtggcctgaagaaccggagctccgagggaccctctagccctagcggcaagctgatgtccccaaagctgtacgtgtgggccaaggatagaccagagatctgggagggagagccaccatgcctgcctccaagggacagcctgaatcagtccctgtctcaggatctgaccatggcccccggctctacactgtggctgagctgtggagtgccacctgacagcgtgtcccggggccctctgagctggacccacgtgcacccaaagggccccaagtccctgctgtctctggagctgaaggacgatcgccctgcccgggacatgtgggtcatggagacaggcctgctgctgccacgcgccacagcacaggatgccggcaagtactattgccaccggggcaacctgaccatgagcttccacctggagatcacagccagacccgtgctgtggcactggctgctgaggaccggaggatggaaggtgtccgccgtgaccctggcatacctgattttctgtctgtgctccctggtgggcatcctgcacctgtgacgtcgacgcgaattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttgg

SEQ ID NO: 20 - DNA, 9229 bp, synthetic construct
  source 1..9229: mol_type = other DNA
ccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattctggccaccatgaaactgcccgtgagactgctggtgctgatgttttggattcccgcttcctcgagccaggtgcagctgaaagagtccggcggaggactggtgcagcctggcggatctctgaagctgagctgtgctgccagcggcttcaccttcagcagctacggcatgagctgggtgcgacagaccgccgacaagagactggaactggtggctaccaccaacaacaacggcggcgtgacctactaccccgacagcgtgaagggcagattcaccatctccagagacaacgccaagaacaccctgtacctgcagatgagcagcctgcagagcgaggacaccgccatgtactactgcgccagatacggctactacgccatggattactggggccagggcatcagcgtgaccgtgtctagcggaggcggcggatctggcggagggggatctagtggcggaggctctgacgtgctgatgacccagacacctctgagcctgccagtgtccctgggcgaccaggccagcatcagctgtagaagcagccagagcatcgtgcacagcaacggcaacacctacctggaatggtatctgcagaagcccggccagagccccaagctgctgatctacaaggtgtccaacagattcagcggcgtgcccgacagattctccggcagcggctctggcaccgacttcaccgtgaagatctccagggtggaagccgaggacctgggcgtgtactactgttttcaaggcagccacgtgccctacaccttcggcggaggcaccaagctggaaatcaaggagagcaagtacggcccaccctgccctccatgtccattttgggtgctggtggtggtgggaggcgtgctggcctgttattctctgctggtgaccgtggccttcatcatcttttgggtgcgcagcaagcggagccggctgctgcactccgactacatgaacatgaccccaagacggcccggaccaacaaggaagcactaccagccttatgcaccaccccgcgattttgcagcataccggagcaagagaggcaggaagaagctgctgtatatcttcaagcagcccttcatgcggcccgtgcagaccacacaggaggaggacggctgctcctgtaggttccctgaagaggaggagggaggatgcgagctgagagtgaagttttctaggagcgccgatgcaccagcataccagcagggacagaatcagctgtataacgagctgaatctgggcaggcgcgaggagtacgacgtgctggataagaggagaggacgggaccccgagatgggaggcaagccaaggcgcaagaacccccaggagggcctgtacaatgagctgcagaaggacaagatggccgaggcctatagcgagatcggcatgaagggagagcggagaaggggcaagggacacgatggcctgtaccagggcctgtccaccgccacaaaggacacctatgatgccctgcacatgcaggccctgcctccaaggggcagcggagccacaaacttctccctgctgaagcaggccggcgatgtggaggagaatcctggaccagtcgacgaattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttgg

SEQ ID NO: 21 - DNA, 22 bp, synthetic construct
  source 1..22: mol_type = other DNA
gctgtcatctcttgtgggctgt

SEQ ID NO: 22 - DNA, 21 bp, synthetic construct
  source 1..21: mol_type = other DNA
actcatgggagctgctggttc

SEQ ID NO: 23 - DNA, 19 bp, synthetic construct
  source 1..19: mol_type = other DNA
cgcacggcaagaggcgagg

SEQ ID NO: 24 - DNA, 20 bp, synthetic construct
  source 1..20: mol_type = other DNA
cactcccaacgaagacaaga

SEQ ID NO: 25 - DNA, 20 bp, synthetic construct
  source 1..20: mol_type = other DNA
tctggtttccctttcgcttt

SEQ ID NO: 26 - DNA, 25 bp, synthetic construct
  source 1..25: mol_type = other DNA
tctctagcagtggcgcccgaacagg

SEQ ID NO: 27 - DNA, 21 bp, synthetic construct
  source 1..21: mol_type = other DNA
aaaacgagcagtgacgtgagc

SEQ ID NO: 28 - DNA, 20 bp, synthetic construct
  source 1..20: mol_type = other DNA
ttcagtcatgctgctagcgc

SEQ ID NO: 29 - DNA, 25 bp, synthetic construct
  source 1..25: mol_type = other DNA
tgcacggaagcgtctcgtctcagtc

SEQ ID NO: 30 - DNA, 122 bp, synthetic construct
  source 1..122: mol_type = other DNA
atcaaaagggctgggggagtctcgcaaggataagaaatagcggttagcgggccgggcggggggtcgggggtctgcgacggagtcctccaaaggctctcaggtggtggtcatcgagatggagc

SEQ ID NO: 31 - DNA, 122 bp, synthetic construct
  source 1..122: mol_type = other DNA
gagctctctttgtgtttccttcaggtttgtaaactcttgagggacgtaagacaatgcatatcaccactaaagcaagcgcagccagggtggtaactccaaagacagttttcagccatgtcttc

SEQ ID NO: 32 - DNA, 138 bp, synthetic construct
  source 1..138: mol_type = other DNA
gtggaacctggtttcttctcacccttcttcatcccactcttattttggcacagcagtccaacgtagatgaattgggatgcagccaccttggtcagtcctatgagtctagagatgtctggaagccagaaccatgtcaaa

SEQ ID NO: 33 - DNA, 22 bp, synthetic construct
  source 1..22: mol_type = other DNA
ctccatcactaggggttccttg

SEQ ID NO: 34 - DNA, 18 bp, synthetic construct
  source 1..18: mol_type = other DNA
gtagataagtagcatggc

SEQ ID NO: 35 - DNA, 17 bp, synthetic construct
  source 1..17: mol_type = other DNA
tagttaatgattaaccc

SEQ ID NO: 36 - DNA, 21 bp, synthetic construct
  source 1..21: mol_type = other DNA
ggttgtgctggtccagggcgt

SEQ ID NO: 37 - DNA, 23 bp, synthetic construct
  source 1..23: mol_type = other DNA
ccaacaaagtgccctactacatc

SEQ ID NO: 38 - DNA, 23 bp, synthetic construct
  source 1..23: mol_type = other DNA
ccgagctgtaccagagcctggcc

SEQ ID NO: 39 - DNA, 20 bp, synthetic construct
  source 1..20: mol_type = other DNA
ctccaagcagatgcagcaga

SEQ ID NO: 40 - DNA, 20 bp, synthetic construct
  source 1..20: mol_type = other DNA
atagccttgcgcatcatggt

SEQ ID NO: 41 - DNA, 22 bp, synthetic construct
  source 1..22: mol_type = other DNA
ccgtggtgctgatgggcaagaa

(19) United States
(12) Patent Application Publication
(10) Pub. No.: US 2026/0053923 A1
(43) Pub. Date: Feb. 26, 2026

(54) Immunotherapy of Skeletal Myopathies Using ANTI-FAP CAR-T Cells

(71) Applicants: Genethon, Evry-Courcouronnes (FR); Institut National de la Santé et de la Recherche Médicale, Paris (FR); Universite Evry Val D'Essonne, Evry-Courcouronnes (FR)

(72) Inventors: Anne Galy, Fontainebleau (FR); Isabelle Richard, Corbeille-Essonnes (FR); Céline Rocca, Ecublens (CH); Sonia Albini, Antony (FR); Maxime Ferrand, Mennecy (FR)

(73) Assignees: Genethon, Evry-Courcouronnes (FR); Institut National de la Santé et de la Recherche Médicale, Paris (FR); Universite Evry Val D'Essonne, Evry-Courcouronnes (FR)

(21) Appl. No.: 19/104,075

(22) PCT Filed: Oct. 9, 2023
(86) PCT No.: PCT/EP2023/077925

§ 371 Date: Feb. 14, 2025

(30) Foreign Application Priority Data
Oct. 7, 2022 (EP) 22306515.2

(51) Int. Cl. A61K 40/11; A61K 35/17; A61K 40/15; A61K 40/31; A61K 40/42; A61K 48/00; A61P 21/00; C07K 14/705; C07K 14/725; C07K 16/40; C12N 5/10; C12N 15/86

(52) U.S. Cl. CPC A61K 40/11; A61K 35/17; A61K 40/15; A61K 40/31; A61K 40/4247; A61K 48/00; A61P 21/00; C07K 14/7051; C07K 14/70521; C07K 16/40; C12N 5/10; C12N 15/86; C07K 2317/53; C07K 2317/56; C07K 2317/622; C07K 2319/03; C12N 2740/15043

(57) ABSTRACT

The invention relates to an immune cell engineered to express a chimeric antigen receptor (CAR) which specifically binds Fibroblast Activation Protein (FAP) for use in the treatment of skeletal muscle fibrosis in muscular dystrophies.

### FIELD OF THE INVENTION

The invention pertains to the field of immunotherapy. The invention relates to the use of chimeric antigen receptor (CAR) immune cells specific for Fibroblast Activation Protein in the treatment of fibrosis in skeletal myopathies, in particular for treating skeletal muscle fibrosis associated with muscular dystrophies. The invention also relates to the use of said CAR immune cells in combination with gene therapy for the treatment of muscular dystrophies.

### BACKGROUND OF THE INVENTION

Skeletal muscle fibrosis is a common hallmark of chronic skeletal muscle degenerative disorders, most prominently associated with the muscular dystrophies and aging. In muscle, fibrosis manifests as the replacement of myofibers with fibroblasts and extracellular matrix components. Excessive accumulation of fibrous tissue within muscle fibers results in reduced muscle contractility and ultimately reduces its ability to regenerate and function.

In addition to worsening the general condition of patients, fibrosis can also impede the efficacy of gene therapy approaches consisting of delivery of the functional gene, particularly via viral vectors. Reducing fibrosis may improve the phenotype of patients with muscular dystrophy, also should make muscle more permissive to viral vectors and improve the efficacy of gene therapy.

To date there is no approved therapy for fibrosis in skeletal myopathies, in particular muscular dystrophies.

Fibroblast activation protein (FAP) was identified as a target for epithelial cancer diagnostic and treatment as it is highly expressed in stromal fibroblasts of the majority of primary and metastatic epithelial tumors while it is generally absent from normal adult tissues. Multiple monoclonal antibodies have been raised against FAP for research, diagnostic and therapeutic purposes (WO 2012/02006). Chimeric antigen receptor (CAR) T cells specific for FAP were also generated and shown to deplete FAP+ stromal cells and reduce tumor growth in mice (WO 2014/055442).

More recently, CAR-T cells specific for FAP were shown to reduce cardiac fibrosis in angiotensin II-induced model of cardiac fibrosis (Aghajanian et al., Nature, 2019, 573, 430-433; WO 2019/067425).

While the presence of the FAP protein in cancer and cardiac fibrosis is well documented, the relative levels of expression of FAP in muscular dystrophies and compared to other markers are not known, and therefore it is not clear that FAP can be a possible target of immunotherapy of muscular dystrophies.

Furthermore, to the inventors' knowledge, the use of a CAR-T cell approach for reducing fibrosis in a skeletal muscle disease has never been reported before.

In addition, the efficacy of CAR-T cells in vivo in both humans and mice, whether treating solid or liquid targets (tumors or other), is highly dependent on the tissue microenvironment and accessibility of the target tissue. It has been shown that in the case of tumors, the effect of CAR-Ts is limited by the immunosuppressive microenvironment around the tumor, notably the presence of regulatory T lymphocytes and macrophages which inhibit the cytolytic activity of CAR-Ts (Sterner and Sterner, Blood, Cancer Journal, 2021, 11, 69). In muscular dystrophies, such as Duchenne muscular dystrophy, the degeneration of muscle fibers creates a significant inflammatory context, with the presence of macrophage cells that secrete TGF beta (an immunosuppressive cytokine) and other cytokines, as well as regulatory T lymphocytes (Li et al., Frontiers in Immunology, March 2018, doi: 10.3389/fimmu.2018.00585; Juban et al., Cell Reports, 2018, 25, 2163-2176). This immunosuppressive microenvironment has not been reported in the case of induced cardiac fibrosis, and presumably is not present considering the short time frame of induction of cardiac fibrosis by pharmacological agents in the study of Aghajanian et al., Nature, 2019, 573, 430-433 and WO 2019/067425.

### SUMMARY OF THE INVENTION

The inventors have generated mRNA expression data in a murine model of Duchenne muscular dystrophy (the severe DBA2 Mdx model) to compare the expression of several fibrosis-associated genes including FAP. The dystrophic mice were compared to their age-matched littermates (WT) over time (2, 3 or 4 months of age). The results show that the FAP gene is over-expressed in skeletal muscles of dystrophic mice at all time points compared to WT controls. The inventors have tested the therapeutic effect of anti-FAP CAR-T cells in muscular dystrophies. They have shown that anti-FAP CAR-T cells not only reduced skeletal muscle fibrosis but also improved the structure of skeletal muscle fiber at the histological level in a DBA2-mdx mouse model of Duchenne muscular dystrophy.

The inventors have also tested combined FAP-CAR-T treatment and AAV-microdystrophin gene therapy in DMD mouse model. Results show that FAP-CAR-T cells mediated fibrosis reduction allow to improve the level of AAV vector copies in the muscle, to improve microdystrophin expression levels in muscle fibers in skeletal muscles in DBA2-mdx mice. DBA2-mdx mice treated with the combined therapy show a decrease in collagen in skeletal muscle as well as an improvement in the structural organization of skeletal muscle fibers. These results provide a novel therapeutic option to treat skeletal muscle fibrosis in muscular dystrophies. The results also show that FAP-CAR-T cell treatment can relieve a block in AAV transduction and provide greater levels of transduction in muscle, possibly enabling a dose reduction for similar effects.

It was not obvious that anti-FAP CAR-T cells would work and significantly reduce fibrosis in muscle dystrophies because unlike induced cardiac fibrosis, fibrosis in muscle dystrophies is characterized by an inflammatory microenvironment which is known to reduce the antitumor efficacy of CAR-T cells.

Therefore, the invention relates to an immune cell engineered to express a chimeric antigen receptor (CAR) which specifically binds Fibroblast Activation Protein (FAP), for use in the treatment of skeletal muscle fibrosis in muscular dystrophies.

In some embodiments of the immune cell for use according to the invention, the CAR comprises: (i) an extracellular domain comprising at least one antigen-binding domain that specifically binds FAP or said other protein, (ii) a transmembrane domain, and (iii) an intracellular domain comprising an intracellular signaling domain capable of activating an immune cell, and optionally comprising one or more co-stimulatory signaling domains.

In some particular embodiments of the immune cell for use according to the invention, the antigen-binding domain is a single-chain variable fragment (scFv) of a monoclonal antibody that specifically binds FAP. In some more particular embodiments, the single-chain variable fragment (scFv) that binds FAP comprises a heavy chain variable domain comprising the amino acid sequence SEQ ID NO: 1 and a light chain variable domain comprising the amino acid sequence SEQ ID NO: 2; preferably comprising the amino acid sequence SEQ ID NO: 8.

In some particular embodiments of the immune cell for use according to the invention, the extracellular domain further comprises a hinge domain, preferably from IgG4 heavy chain.

In some particular embodiments of the immune cell for the use according to the invention, the transmembrane domain is from CD28.

In some particular embodiments of the immune cell for the use according to the invention, the intracellular signaling domain is a CD3 zeta signaling domain.

In some particular embodiments of the immune cell for the use according to the invention, the intracellular domain further comprises one or more co-stimulatory signaling domains, preferably both CD28 and 4-1BB co-stimulatory signaling domains.

In some preferred embodiments of the immune cell for the use according to the invention, the CAR comprises from its N- to C-terminus: a signal peptide from mouse Ig-kappa light chain; a scFv fragment from an anti-FAP monoclonal antibody, a modified hinge domain from IgG4 heavy chain; a transmembrane domain from human CD28, a first co-stimulatory domain from human CD28, a second co-stimulatory domain from human 4-1BB and an intracellular signaling domain from human CD3 zeta chain; preferably the CAR comprises the amino acid sequence SEQ ID NO:16.

In some particular embodiments, the immune cell for use according to the invention is a lymphocyte such as T cell and/or NK cell, preferably a cytolytic lymphocyte such as a cytolytic T cell.

In some particular embodiments, the immune cell for use according to the invention is modified with an expression vector comprising a nucleic acid construct encoding the CAR, preferably chosen from a lipid nanoparticle packaging an RNA molecule and a lentiviral vector, more preferably a self-inactivating and/or VSVG-pseudotyped lentiviral vector.

In some particular embodiments, the immune cell for use according to the invention reduces the expression level of at least one biomarker of fibrosis; preferably Collagen type III.

In some particular embodiments, the immune cell for use according to the invention is for use in combination with a vector for gene therapy of muscular dystrophies, preferably a recombinant AAV vector. In some more particular embodiments, the combination therapy comprises the administration of a reduced dose of vector for gene therapy compared to the use of the vector for gene therapy without the immune cell.

In some particular embodiments, the immune cell for use according to the invention is for the treatment of muscular dystrophies chosen from Dystrophynopathies, Limb-girdle muscular dystrophies and Congenital muscle dystrophies; preferably Duchenne muscular dystrophy.

### DETAILED DESCRIPTION OF THE INVENTION

The invention provides an immunotherapeutic approach for treating skeletal muscle fibrosis in muscular dystrophies, using chimeric antigen receptor (CAR) immune cells specific for Fibroblast Activation Protein (FAP) that can recognize and deplete the fibrotic cells in skeletal muscle.

### Chimeric Antigen Receptor (CAR)

Chimeric antigen receptors (CARs) are well-known in the art (Review for example in; Mehrabagi et al., Biomedicine and Pharmacotherapy, 2022, 146; Rafiq et al., Nat. Rev. Clinical Oncology, 2020, 17, 150-). CARs are synthetic antigen receptors that generally incorporate an extracellular antigen-binding domain with intracellular signaling motifs involved in lymphocyte activation (Sadelain et al., Nat. Rev. Cancer, 2003, 3, 35-45). CARs comprise three major domains: an extracellular domain, a transmembrane domain, and an intracellular domain. The extracellular domain comprises a target antigen-binding region (FAP-binding region in the present invention) and is responsible for antigen recognition. The extracellular domain optionally comprises a signal peptide (SP) so that the CAR can be glycosylated and anchored in the cell membrane of the immune effector cell via its transmembrane domain. The optional signal peptide is at the N-terminus of the extracellular domain. The transmembrane domain connects the extracellular domain to the intracellular domain and resides within the cell membrane when expressed by a cell. An optional hinge domain consisting of a flexible linker that provides flexibility to the binding domain may be inserted between the extracellular domain and the transmembrane domain. The intracellular domain of the CAR transmits an activation signal to the immune effector cell after antigen recognition and usually comprises an intracellular signaling domain (activation domain) and optionally one or more co-stimulatory signaling domains (co-stimulatory domains). An intracellular signaling domain generally contains immunoreceptor tyrosine-based activation motifs (ITAMs) that activate a signaling cascade when the ITAM is phosphorylated. The term “co-stimulatory signaling domain” refers to intracellular signaling domain(s) from costimulatory protein receptors, such as CD28, 4-1BB, ICOS and others, that are able to enhance T-cell activation by T-cell receptors. The different domains are linked to each other by a peptide bond or linker.

CARs have been categorized into fifth distinct generations depending on the structure of the intracellular domain. The first generation of CARs only contain the CD3 chain from the CD3 TCR (CD3 zeta or CD35) or Fc receptor chain (FcR), but the second generation includes an additional costimulatory signaling domain (CD28 or 4-1BB). The third-generation CARs were generated by combining multiple co-stimulatory domains, such as CD28-41BB or CD28-OX40, to enhance CAR-T cells potency. The fourth generation, also called T cell redirected for universal cytokine-mediated killing or TRUCKs, was generated by further genetic modification, including additional transgenes for cytokine secretion (e.g., IL-2, IL-5, IL-12) or additional co-stimulatory ligands, to the base of the second-generation constructs. The fifth generation CAR-T cells contain an extra intracellular domain than their predecessors. The CARs comprise truncated intracellular domains of cytokine (e.g., IL-2R chain fragment) with a motif for binding transcription factors such as STAT-3/5.

Fibrosis, also known as fibrotic scarring, is a pathological process resulting generally from chronic inflammation and in which connective tissue replaces normal parenchyme tissue, leading to considerable tissue remodeling and the formation of permanent scar tissue. Fibrosis is defined as excessive extracellular matrix deposition that leads to tissue destruction and impairment of organ function. Fibrosis is characterized by the activation and proliferation offibroblasts and the accumulation of extracellular matrix components including collagen and glycosaminoglycans into the surrounding connective tissue, in response to injury, infection or other known or unknown disease processes. In fibrosis, the accidental excessive accumulation of extracellular matrix components, such as the collagen produced by fibroblasts, leads to the formation of a permanent fibrotic scar.

The term “overexpressed in muscular dystrophies” as used herein refers to a FAP protein encoded by a FAP gene having an expression level in skeletal muscles that is increased in subjects suffering from muscular dystrophies compared to normal subjects. The gene expression level is preferably increased by at least 1.5 fold; in particular 1.5; 2; 2.5; 3 to 20 folds, in skeletal muscles from subjects suffering from muscular dystrophies compared to normal subjects. Gene expression levels may be measured in mouse models of muscular dystrophies that are well-known in the art such as the DBA2-mdx mice disclosed in the examples.

As used herein, the term “gene expression level” refers to an amount or a concentration of a transcription product (or transcript), for instance mRNA, or of a translation product, for instance a protein or polypeptide. Typically, a level of mRNA expression can be expressed in units such as transcripts per cell or nanograms per microgram of tissue. A level of a polypeptide can be expressed as nanograms per microgram of tissue, for example. Alternatively, relative units can be employed to describe a gene expression level. Typically, gene expression levels may be determined according to the routine techniques, well-known of the person skilled in the art and disclosed in the examples of the present application. Methods for determining the quantity of mRNA are well known in the art. For example, the mRNA contained in the sample is first extracted according to standard methods, for example using lytic enzymes or chemical solutions or extracted by nucleic-acid-binding resins following the manufacturer's instructions. The extracted mRNA present in the sample is then detected by any suitable method such as with no limitations: spectrophotometric methods; Hybridization such as Northern Blotting, Microarray, in situ hybridization such as RNAscope; Sequencing such as next generation sequencing (NGS) and Single-molecule sequencing; micro and nanosensor-based electrochemical, electrical, mechanical or optical detection and Nucleic acid amplification techniques. Nucleic acid amplification methods include isothermal and polymerase chain reaction (PCR)-based techniques such as for example, reverse transcription-PCR (RT-PCR), quantitative PCR (Q-PCR) in particular real time Q-PCR, RT-qPCR, droplet digital PCR (ddPCR), PCR-HM (High Resolution DNA Melting, PCR coupled to ligase detection reaction based on fluorescent microspheres (Luminex® microspheres). RNA-Seq (RNA-sequencing) also called whole transcriptome shotgun sequencing (WTSS) is a technique that can examine the quantity and sequences of RNA in a sample using next generation sequencing (NGS) (Review in Wang et al., Nat. Rev. Genet., 2009, 10, 57-63). It analyzes the transcriptome of gene expression patterns encoded within RNA. The level of the protein may be determined by any suitable methods known by skilled persons. Usually, these methods comprise contacting a cell sample, preferably a cell lysate, with a binding partner capable of selectively interacting with the protein present in the sample. The binding partner is generally a polyclonal or monoclonal antibody, preferably monoclonal. The methods generally include suitable labels such as fluorescent, chemiluminescent, radioactive, enzymatic labels or dye molecules, or other methods for detecting the amount of complex formed between the protein and the antibody or antibodies reacted therewith. The quantity of the protein may be measured, for example, by semi-quantitative Western blots, enzyme-labelled and mediated immunoassays, such as ELISAs, biotin/avidin type assays, radioimmunoassay, immune-electrophoresis, mass spectrometry, immunoprecipitation or by protein or antibody arrays.

The term “muscular dystrophies” as used herein refer to muscular dystrophies which affect skeletal muscles, in particular muscular dystrophies which primarily affect skeletal muscles. Muscular dystrophies include muscular dystrophies that affect essentially skeletal muscles; muscular dystrophies that affect exclusively skeletal muscles; muscular dystrophies that do not affect cardiac muscle (heart) and/or muscular dystrophies that are not associated with a heart disease (cardiomyopathy).

The term “Fibroblast Activation Protein” (Fibroblast Activation Protein Alpha, Prolyl Endopeptidase FAP or FAP), also known as Seprase, refers to a protein encoded by the FAP gene in a mammalian genome. FAP is a homodimeric cell surface glycoprotein belonging to the serine protease family. Representative examples of FAP include without limitation, human (NCBI Gene ID: 2191), mouse (NCBI Gene ID: 14089) and other functional orthologs thereof. Human FAP has the 760 amino acid sequence UniProtKB/Swiss-Prot Q12884. Mouse FAP has the 761 amino acid sequence UniProtKB/Swiss-Prot P97321.

The term “functional ortholog” as used herein, refers to a gene from another species which encodes a protein having substantially the same activity than that of the initial gene. In the following description, the amino acid residues are designated by the standard one letter amino acid code.

“a”, “an”, and “the” include plural referents, unless the context clearly indicates otherwise. As such, the term “a” (or “an”), “one or more” or “at least one” can be used interchangeably herein; unless specified otherwise, “or” means “and/or”.

As with other CARS, the CARs used in the present invention comprise an extracellular domain, a transmembrane domain, and an intracellular domain. The extracellular domain comprises an antigen-binding domain that can specifically bind FAP The antigen-binding domain may be derived from any ligand of FAP that binds FAP on cells and/or that binds the extracellular region of FAP. The ligand (anti-FAP binding agent) may be a natural ligand, a synthetic ligand, or a variant and/or fragment thereof capable of specifically binding FAP. Natural ligands include antibodies specific for FAP (anti-FAP antibodies) and antigen-binding fragments thereof. Synthetic ligands include for example, oligonucleotides and peptides such as aptamers, and DARPins (designed ankyrin repeat proteins) that may be selected by screening a library of random oligonucleotide or peptide sequences for their ability to specifically bind FAP by standard assays that are well-known in the art.

A specified ligand specifically binds to (or is specific for) its particular target (FAP) when it does not bind in significant amount (detectably bind) to other proteins present in the sample or to other proteins to which the ligand may come into contact in an organism, while detectably binding to its target. The specific binding of a ligand to its target may be determined by standard assays such as immunoassay in the case of an antibody. A ligand specifically binds to its target when it has a dissociation constant (KD) of 1 μM or less for its target in a standard KD determination assay. KD values are expressed as molar concentration (M); KD for antibodies are usually determined by Surface plasmon resonance using Biacore assay.

By the term “specifically binds,” as used herein with respect to an antibody, is meant an antibody or antibody fragment, which recognizes and binds with a specific antigen, but does not substantially recognize or bind other molecules in a sample. For example, an antibody that specifically binds to an antigen from one species may also bind to that antigen from one or more species. But, such crossspecies reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” or “specifically binds,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies.

The term “antibody fragment”, as used herein, refers to a fragment of an antibody that retain the ability to specifically bind an antigen (e.g., FAP). Examples of binding fragments encompassed within the term “antigen-binding fragment” of an antibody include a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., 1989 Nature 341:544-546), which consists of a VH domain; a nanobody, camelid VHH single-domain antibody fragment, and other fragments and any fusion proteins comprising such antigen-binding fragments. Furthermore, although the two domains of the Fv fragment, VL and VH, are coded for by separate genes, they can be joined, using recombinant methods, by a synthetic linker that enables them to be made as a single chain protein in which the VL and VH regions pair to form a monovalent molecule (known as single chain variable fragment or scFv); see e.g., Bird et al., 1988 Science 242:423-426; and Huston et al., 1988 Proc. Natl. Acad. Sci. 85:5879-5883). Suitable linkers for scFv are known in the art and include for example linkers comprising Glycine and Serine residues and others. Such single chain antibodies are also intended to be encompassed within the term “antigen-binding fragment” of an antibody. These antibody fragments are obtained using conventional techniques known to those of skill in the art.

The term “monoclonal antibody” as used herein refers to a preparation of antibody molecules of single specificity. A monoclonal antibody displays a single binding specificity and affinity for a particular epitope. Accordingly, the term “human monoclonal antibody” refers to an antibody displaying a single binding specificity which has variable and constant regions derived from or based on human germline immunoglobulin sequences or derived from completely synthetic sequences. The method of preparing the monoclonal antibody is not relevant for the binding specificity.

As used herein, the term “recombinant antibody” refers to antibodies which are produced, expressed, generated or isolated by recombinant means, such as antibodies which are expressed using a recombinant expression vector transfected into a host cell; antibodies isolated from a recombinant combinatorial antibody library; antibodies isolated from an animal (e.g. a mouse) which is transgenic due to human immunoglobulin genes; or antibodies which are produced, expressed, generated or isolated in any other way in which particular immunoglobulin gene sequences (such as human immunoglobulin gene sequences) are assembled with other DNA sequences. Recombinant antibodies include, for example, chimeric and humanized antibodies.

Various anti-FAP antibodies and derived CARs have been described in the art and can be used in the CARs according to the invention. Non-limiting examples of known anti-FAP antibodies including anti-human or murine FAP antibodies, as well as chimeric, fully human and humanized versions thereof and derived CARs are disclosed in the following references. The well-characterized F19 anti-human FAP monoclonal antibody (ATCC number HB 8269) is described in WO 93/05804. Sibrotuzumab (BIBHB1) is a humanized version of the F19 antibody that specifically binds human FAP as described in WO 99/57151. WO 99/57151 discloses humanized version of the F19 antibody that specifically binds human FAP and comprising the variable light chain region (VL) of SEQ ID NO: 2 or 20 and the variable heavy chain region (VH) of SEQ ID NO: 8, 10, 12, 14 or 22 (see also FIGS. 24 and 25). Further humanized or fully human antibodies against the FAP antigen with F19 epitope specificity were developed (described in Mersmann et al., Int. J. cancer, 2001, 92, 240-248; Schmidt et al., Eur. J. Biochem., 2001, 268, 1730-1738; WO 01/68708). The OS4 antibody is another humanized (CDR-grafted) version of the F19 antibody (Wüest et al., J. Biotech., 2001, 92, 159-168), while scFv MO33 and scFv MO36 have a different binding specificity from F19 and are cross-reactive for the human and mouse FAP proteins (Brocks et al., Mol. Med., 2001, 7, 461-469). The VH, VL and 6CDR sequences of scFv MO33 and scFv MO36 are disclosed in FIG. 2 of Brocks et al. (Mol. Med; 2001, 7, 461-469). Second generation CAR derived from scFv MO36 is disclosed in Kakarla, Mol. Ther., 2013, 21, 1611-1620. Other anti-human or murine FAP antibodies, as well as chimeric, fully human and humanized versions thereof are described in WO 2007/077173; Ostermann et al., Clin. Cancer Res. 2008, 14, 4584-4592; WO 2012/020006; WO 2014/055442; Wang et al., Cancer Immunol. Res., 2014, 2, 154-166). In particular, Ostermann et al., Clin. Cancer Res. 2008 discloses the monoclonal antibody FAP5-DM1 that targets a shared epitope of human, mouse and cynomolgus monkey FAP-alpha. Third generation CAR-T cells derived from mAb FAP5 or Sibrotuzumab are disclosed in Tran et al., J. Exp. Med., 2013, 210, 1125-1135. An anti-mouse FAP antibody (clone 73.3) is described in WO 2014/055442 and Wang et al., Cancer Immunol. Res., 2014, 2, 154-166). A review of FAP-targeted CAR-T is provided in Bughda et al., Immunotargets Ther., 2021, 313-323.

Alternatively, new anti-FAP antibodies may be obtained by standard methods that are well-known in the art. Antibodies of the present disclosure can be produced by immunization of a laboratory animal with an antigen (FAP protein or fragment thereof, eventually coupled to a carrier) to induce the production of anti-FAP antibodies by the B cells of said mammal; and recovery of the antibodies from the serum of the immunized animal. To obtain monoclonal antibodies, B cells are isolated from the spleen of the immunized animals and immortalized according to standard hybridoma production techniques. Antibodies of the present disclosure can also be obtained by screening of a phage display library. In particular, VH and VL fragments of the anti-FAP antibodies may be screened from a phage display library using a peptide antigen and recombinant antibodies may be produced according to standard techniques which are well-known in the art. Antibody VH and VL sequences may be obtained by single-cell B-cell receptor sequencing using high throughput sequencing techniques as disclosed in Goldstein et al., Communications Biology, 2019, 2, 304. Antibodies of the present disclosure can also be produced in a host cell transfectoma using, for example, a combination of recombinant DNA techniques and gene transfection methods as is well known in the art (Morrison, Science, 1985, 229, 1202-1207). For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. When recombinant expression vectors encoding antibody genes are introduced into host cells, in particular eukaryotic cells such as mammalian cells, the antibodies are produced by culturing the host cells for a period of time sufficient for expression of the antibody in the host cells and, optionally, secretion of the antibody into the culture medium in which the host cells are grown. Antibodies can be recovered and purified for example from the culture medium after their secretion using standard protein purification methods (Shukla et al., Journal of Chromatography, 2007, 848, 28-39). In some embodiments, the antigen-binding domain is an antibody fragment that specifically binds FAP, in particular a Fab, nanobody, or single-chain variable fragment (scFv). The antibody or antibody fragment may specifically bind human FAP; may specifically bind mouse FAP; or may be cross-reactive for the human and murine FAP proteins. In some embodiments, the antibody or antibody fragment specifically binds human FAP or is cross-reactive for the human and murine FAP proteins.

In some particular embodiments, the antigen-binding domain is a scFv comprising a variable heavy (VH) domain and a variable light (VL) domain having one to six CDR sequences (VH-CDR1, VH-CDR2, VH-CDR3, VL-CDR1, VL-CDR2, VL-CDR3) from the scFv comprising a variable heavy (VH) domain comprising the amino acid sequence SEQ ID NO:1 and a variable light (VL) domain comprising the amino acid sequence SEQ ID NO: 2. The scFv comprising the VH of SEQ ID NO: 1 and the VL of SEQ ID NO: 2 corresponds to the clone 73.3 described in WO 2014/055442 and has VH-CDR1 sequence of SEQ ID NO: 3, VH-CDR2 sequence of SEQ ID NO: 4, VL-CDR1 sequence of SEQ ID NO: 5, VL-CDR2 sequence of SEQ ID NO: 6 and VL-CDR3 sequence of SEQ ID NO: 7. In some preferred embodiments, the scFv comprises a variable heavy (VH) domain comprising the amino acid sequence SEQ ID NO: 1 and a variable light (VL) domain comprising the amino acid sequence SEQ ID NO: 2 or functional variants of said VH and VL sequences. In some preferred embodiments, the scFv comprises the amino acid sequence SEQ ID NO: 8 or a functional variant thereof.

In some particular embodiments, the antigen-binding domain is from a monoclonal antibody that binds to human and murine FAP. The antibody or antibody fragment may comprise one to six of the CDRs chosen the sequences SEQ ID NO: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 11, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175 and 177 as disclosed in WO 2012/020006. In some specific embodiments, the antibody comprises an antibody heavy chain variable region or domain (VH) and/or an antibody light chain variable region or domain (VL), particularly both a VH and a VL, selected from the following sequences: SEQ ID NO: 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309 and 311 as disclosed in WO 2012/020006.

As used herein, the term “variant” refers to a polypeptide comprising an amino acid sequence having at least 70% sequence identity with the native sequence. The term “variant” refers to a functional variant having the activity of the native sequence. The activity of a variant or fragment may be assessed using methods well-known by the skilled person such as immunoassays with FAP protein for monoclonal antibodies or antigen-binding fragment thereof.

In particular, it is contemplated that monoclonal antibodies or antigen-binding fragment thereof may have 1, 2, 3, 4, 5, 6, or more alterations (i.e., mutations) in the amino acid sequence of 1, 2, 3, 4, 5, or 6 CDRs of monoclonal antibodies provided herein. The amino acid mutation may be an insertion, deletion, or substitution with a conserved or non-conserved amino acid. In some particular embodiments, the monoclonal antibodies or antigen-binding fragment have 1 or 2 conservative substitutions in the amino acid sequence of 1, 2, 3, 4, 5, or 6 CDRs of monoclonal antibodies provided herein. Examples of conservative substitutions are within the groups of basic amino acids (arginine, lysine and histidine), acidic amino acids (glutamic acid and aspartic acid), polar amino acids (glutamine and asparagine), hydrophobic amino acids (methionine, leucine, isoleucine and valine), aromatic amino acids (phenylalanine, tryptophan and tyrosine), and small amino acids (glycine, alanine, serine and threonine).

The percent amino acid sequence or nucleotide sequence identity is defined as the percent of amino acid residues or nucleotides in a Compared Sequence that are identical to the Reference Sequence after aligning the sequences and introducing gaps if necessary, to achieve the maximum sequence identity and not considering any conservative substitutions for amino acid sequences as part of the sequence identity. Alignment for purposes of determining percent amino acid sequence identity can be achieved in various ways known to a person of skill in the art, for instance using publicly available computer software such as the GCG (Genetics Computer Group, Program Manual for the GCG Package, Version 7, Madison, Wisconsin) pileup program, or any of sequence comparison algorithms such as BLAST (Altschul et al., J. Mol. Biol., 1990, 215, 403-), FASTA or CLUSTALW. When using such software, the default parameters, are preferably used.

The extracellular domain may comprise or consist of the FAP antigen-binding domain, in particular an anti-FAP antibody scFv fragment as described herein.

In some embodiments, the extracellular domain further comprises a hinge domain. The hinge domain consists of a flexible linker that provides flexibility to the antigen-binding domain. The hinge domain may be a natural or synthetic sequence; it may be derived from any suitable protein. The hinge domain is inserted between the extracellular domain and the transmembrane domain. Examples of hinge domain include the hinge domain from immunoglobulin superfamily of proteins such as Ig (heavy chain), in particular IgG1 and IgG4; CD3, CD4, CD28, and CD8 alpha, and modified hinge domains thereof (Fujiwara et al., Cells, 2020, 9, 1182-; Peters et al., J. Biol. Chem., 2012, 287, 24525-24533). A particular IgG4 hinge domain is a modified human IgG4 hinge domain containing the S225P mutation, corresponding to the sequence SEQ ID NO: 9. In some particular embodiments, the hinge domain is from a molecule selected from the group consisting of: immunoglobulin superfamily of proteins such as IgG4, IgG1, CD28, CD3 and CD8. In some more particular embodiments, the hinge domain is from IgG4 heavy chain; preferably comprising the sequence SEQ ID NO: 9.

The extracellular domain may optionally comprise a signal peptide at its N-terminus. Non-limiting examples of signal peptides are signal peptides from immunoglobulin molecules, in particular Ig-kappa light chain, more particularly the signal peptide of SEQ ID NO: 10. The extracellular domain may also optionally comprise one or more additional antigen-binding domains which specifically bind different antigens. The extracellular domain may comprise at least two antigen-binding domains which specifically bind different FAP proteins according to the present disclosure. Alternatively, the extracellular domain may comprise at least one first antigen-binding domain which specifically binds a FAP protein according to the present disclosure. The additional antigen-binding domain may be an antibody scFv fragment or another ligand such as a cytokine that binds a cytokine receptor present on a target cell. For example, Tandem CAR (TanCAR) systems consist of a single CAR polypeptide comprising two antigen-binding domains that bind to two different antigens from the same target cell to increase safety and/or efficacy of the CAR-T cells.

The transmembrane domain may be derived from any transmembrane protein or may be a synthetic sequence. The transmembrane region may be derived (i.e., comprise at least the transmembrane region(s) of the alpha or beta chain of the T-cell receptor, CD3 zeta, CD28, CD3 epsilon, CD45, CD4, CD5, CD8 (e.g., CD8 alpha (CD8a), CD8 beta), CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFR, HVEM (LIGHTR), SLAMF7, NKp80 (KLRF1), CD160, CD19, IL2R beta, IL2R gamma, IL7R alpha, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAMI (CD226), SLAMF4 (CD244, 2B4), CD84, CD96, CEACAMI, CRTAM, Ly9 (CD229), CD160 (BY55), PSGLI, CD100 (SEMA4D), SLAF6 (NTB-A, Ly108), SLAM (SLAMFI, CD150, IPO-3), BLAME (SLAMF8), SEPLG (CD162), LTBR, PAG/Cpb, and modified versions thereof (e.g., functional variants thereof). Alternatively, the transmembrane domain may be synthetic and comprise predominantly hydrophobic residues such as leucine and valine. The CAR may have more than one transmembrane domain, which can be the repeat of the same transmembrane domain or can be different transmembrane domains. The transmembrane domain may be linked to the intracellular domain via a short sequence, preferably of up to 10 amino acids in length. Examples of transmembrane domain include the transmembrane domain from the CD3 zeta chain, CD8a or CD28 molecule, in particular human CD3 zeta, CD8a or CD28 molecule. A particular example of transmembrane domain from human CD28 comprises the sequence SEQ ID NO: 11. In some embodiments, the transmembrane domain is from a molecule selected from the group consisting of: human CD28 and human CD8a; in particular human CD28 transmembrane comprising the sequence SEQ ID NO: 11.

As with other CARs, the CAR used in present invention comprises a transmembrane domain and an intracellular domain capable of activating an immune effector cell. The intracellular domain is the functional end of the CAR that after antigen recognition transmits a signal to the immune effector cell, activating at least one of the normal effector functions of the immune effector cell. Effector function of a T cell, for example may be cytolytic activity or helper activity including the secretion of cytokines. Immune effector cell activity may be evaluated by standard assays that are well-known in the art such as proliferation assays, cytokine assays and cytotoxicity assays. The intracellular domain may comprise the intracellular signaling domain of a T cell receptor (TCR) and optional co-receptors. The term “co-stimulatory signaling domain” refers to the intracellular signaling domain from a costimulatory molecule. A costimulatory molecule is a cell surface molecule (protein receptor) other than an antigen receptor or their ligands that is required for an efficient response of lymphocytes to an antigen.

Cytoplasmic signaling sequences that regulate primary activation of the TCR complex that act in a stimulatory manner may contain signaling motifs which are known as immunoreceptor tyrosine-based activation motifs (ITAMs). Examples of ITAM containing cytoplasmic signaling sequences include those derived from CD8, CD3 zeta, CD3 delta, CD3 gamma, CD3 epsilon, CD32 (Fc gamma RIIa), DAP10, DAP12, CD79a, CD79b, FcgammaRIgamma (FcγRIγ), FcgammaRIIIgamma (FcγRIIIγ), FcRepsilonRIbeta (FcεRIβ or FCERIB), and FcepsilonRIgamma (FcεRIγ or FCERIG). Example of costimulatory molecules include CD27, CD28, 4-1BB (CD137), OX40, CD30, CD40, ICOS, lymphocyte-function associated antigen-1 (LFA-1), CD2, CD7, LIGHT, NKG2C, B7-H3, KIR2DS2, and a ligand that specifically binds CD123, CD8, CD4, b2c, CD80, CD86, DAP10, DAP12, MYD88, BTNL3 and NKG2D.

T-cell surface glycoprotein CD3 Zeta (CD3ζ) chain, also known as T-cell receptor T3 zeta chain, TCR zeta Chain or CD247 is encoded by the (I) 247 gene (Gene ID 919 in human). Human CD3 zeta protein has the amino acid sequence UniProtKB/Swiss-Prot P20963. T-cell-specific surface Glycoprotein CD28 is encoded by the CD28 gene (Gene ID 940 in human). Human CD28 protein has the amino acid sequence UniProtKB/Swiss-Prot P10747.

4-1BB (CD137) also known as TNF Receptor Superfamily Member 9 or TNFR9 is expressed on the surface of activated T cells and encoded by the TNFRSF9 gene (Gene ID 3604 in human). Human 4-1BB protein has the amino acid sequence UniProtKB/Swiss-Prot Q07011.

The intracellular domain comprises an intracellular signaling domain (activation domain) and optionally one or more co-stimulatory signaling domains (co-stimulatory domains). The co-stimulatory signaling region may contain 1, 2, 3, 4 or more cytoplasmic domains of one or more intracellular signaling and/or costimulatory molecules. The co-stimulatory signaling region may contain one or more mutations in the cytoplasmic domains of the one or more intracellular signaling and/or costimulatory molecules that enhance signaling. While usually the entire signaling domain is used, a truncated signaling domain may be used instead as long as it transduces the effector function signal. The intracellular signaling domain is usually at the C-terminus of the intracellular signaling domain and the CAR and the co-stimulatory signaling domains are at the N-terminus of the intracellular signaling domain.

In some embodiments, the intracellular signaling domain is a CD3 zeta signaling domain; in particular human CD3 zeta signaling domain comprising the sequence SEQ ID NO: 12.

In some embodiments, the intracellular domain further comprises one or more co-stimulatory signaling domains from molecules selected from the group consisting of: CD28 and 4-1BB; preferably from human CD28 or 4-1BB. A particular example of co-stimulatory signaling domain from human CD28 comprises the sequence SEQ ID NO: 13. A particular example of co-stimulatory signaling domain from human 4-1BB comprises the sequence SEQ ID NO: 14. The intracellular domain preferably comprises both CD28 and 4-1BB co-stimulatory signaling domains, more preferably from human CD28 and 4-1BB.

The CAR may be any generation of CAR, preferably other than the first generation, more preferably a third or fourth generation CAR.

The CAR may be any one of the various CAR constructs such as for example a Multi-chain CAR, TRUCK, Universal CAR, Self-driving CAR, Armored CAR, Self-destruct CAR, Conditional CAR, Marked CAR, tandem CAR (TanCAR), Dual CAR, safety CAR (sCAR) and others.

A multi-chain CAR comprises separate extracellular ligand binding and signaling domains in different transmembrane polypeptides. The signaling domains can be designed to assemble in juxta membrane position. For example, the multi-chain CAR can comprise a part of an FCERI alpha chain and a part of an FCERI beta chain such that the FCERI chains spontaneously dimerize together to form a CAR.

Armored CAR are CAR T cells engineered to be resistant to immunosuppression and may be genetically modified to no longer express various immune check-point molecules (for example, cytotoxic T lymphocyte-associated antigen 4 (CTLA-A4) or programmed cell death protein 1 (PD-1), with an immune check-point switch receptor, or may be administered with a monoclonal antibody that blocks immune checkpoint signaling.

A self-destructing CAR may be designed using RNA delivered by electroporation to encode the CAR. Alternatively, inducible apoptosis of the T cell may be achieved based on ganciclovir binding to thymidine kinase gene-modified lymphocytes or a system of activation of human caspase 9 by a small-molecule dimerizer.

A safety CAR (sCAR) consists of an extracellular scFv fused to an intracellular inhibition domain. SCAR T cells co-expressing a standard CAR become activated only when encountering target cells that possess the standard CAR target but lack the sCAR target.

A dual CAR T cell expresses two separate CARs with different ligand binding targets; one CAR includes only the CD3 zeta domain and the other CAR includes only the co-stimulatory domains. Dual CAR T cell activation requires co-expression of both targets.

A tandem CAR (TanCAR T) T cell expresses a single CAR consisting of two linked single-chain variable fragments (scFv) that have different antigen specificities fused to intracellular co-stimulatory domain(s) and a CD3 zeta domain; TanCAR T cell activation is achieved only when target cell co-express both targets.

A conditional CAR T is by default unresponsive or switched off until the addition of a small molecule to complete the circuit, enabling thus transduction of both signal 1 and signal 2, thereby activating the CAR T cell. Alternatively, T cells may be engineered to express an adaptor-specific receptor with affinity for subsequently administered secondary antibodies directed to target antigen.

In some embodiments, the CAR comprises human sequences, e.g.; sequences from human molecules or humanized molecules.

As with other CARS, the CAR used in the present invention may comprise additional sequences.

In some particular embodiments, the CAR comprises from its N- to C-terminus: a signal peptide from mouse Ig-kappa light chain (SEQ ID NO: 10); a scFv fragment from an anti-FAP monoclonal antibody (anti-FAP scFv of SEQ ID NO: 8 comprising the VH of SEQ ID NO: 1 and the VL of SEQ ID NO: 2 joined by the linker of SEQ ID NO: 15), a modified hinge domain from human IgG4 heavy chain (SEQ ID NO: 9); a transmembrane domain from human CD28 (SEQ ID NO: 11), a first co-stimulatory domain from human CD28 (SEQ ID NO: 13), a second co-stimulatory domain from human 4-1BB (SEQ ID NO: 14) and an intracellular signaling domain from human CD3 zeta chain (SEQ ID NO: 12); preferably the CAR comprises the amino acid sequence SEQ ID NO: 16.

The CAR is generally delivered to the immune cells in the form of a nucleic acid construct comprising a nucleotide sequence encoding the CAR as described herein. The nucleic acid construct may comprise or consist of DNA, RNA or a synthetic or semi-synthetic nucleic acid which is expressible in the individual's target cells (immune cells). The sequence encoding the CAR may be codon-optimized for expression in the individual's immune cells, preferably a human individual. Appropriate software for codon optimization in the desired individual are well-known in the art and publicly available. In some embodiments, the nucleotide sequence encoding the CAR comprises the sequence SEQ ID NO: 17. SEQ ID NO: 17 is the nucleotide sequence encoding the CAR amino acid sequence SEQ ID NO: 16.

The nucleic acid construct comprises an expression cassette wherein the coding sequence is operably linked to appropriate regulatory sequences for expression of a transgene in the individual's target cells (immune cells). Such sequences which are well-known in the art include in particular a promoter, and further regulatory sequences capable of further controlling the expression of a transgene, such as without limitation, enhancer; terminator; intron; silencer, in particular tissue-specific silencer such as miRNAs; and post-translational regulatory element.

The promoter may be a tissue-specific, ubiquitous, constitutive or inducible promoter that is functional in the individual's target cells. Examples of constitutive promoters which can be used in the present invention include without limitation: phosphoglycerate kinase promoter (PGK); elongation factor-1 alpha (EF-1 alpha) promoter including the short form of said promoter (EFS); dihydrofolate reductase promoter; β-actin promoter; viral promoters such as cytomegalovirus (CMV) immediate early enhancer and promoter, cytomegalovirus enhancer/chicken beta actin (CAG) promoter, SV40 early promoter and retroviral 5′ and 3′ LTR promoters including hybrid LTR promoters. Preferred ubiquitous promoter is EF-1 alpha promoter. Examples of inducible promoters which can be used in the present invention include Tetracycline-regulated promoters. The promoters are advantageously human promoters, i.e., promoters from human cells or human viruses. Such promoters are well-known in the art and their sequences are available in public sequence data base.

Moreover, transgene expression levels can be improved by the inclusion, in the transgene expression cassette, of post-transcriptional regulatory elements such as the Woodchuck hepatitis virus (WHV) post-transcriptional regulatory element (WPRE), able to increase transcript levels and/or stability.

### Engineered Immune Cell

The immune cell comprise any leukocyte involved in defending the body against foreign invaders. The immune cell can comprise lymphocytes, monocytes, monocytes, macrophages, dendritic cells, mast cells, neutrophils, basophils, eosinophils, or any combinations thereof. Immune cell as used herein includes immune effector cell and precursors thereof. Immune effector cell precursors have greater proliferative capacity. Therefore, the population of engineered immune cells preferably contains effector cells as well as less differentiated cell subsets that have greater proliferative capacity.

In some embodiments, the immune cells are lymphocytes, in particular T cells and/or NK cells. Lymphocytes include T lymphocytes, such as alpha-beta T cells and gamma-delta T cells; Natural Killer (NK) cells; Natural Killer T (NKT) cells; cytokine induced killer cells; cytotoxic T lymphocytes (CTLs); lymphokine activated killer (LAK) cells. Effector function of a lymphocyte such as T cell or NK cells may be a cytolytic activity. Effector function of a T cell may also be a helper activity. Effector function of a T cell (cytolytic or helper) includes the secretion of cytokines. T cells are CD3+ cells. NK cells are CD56+CD3-cells. Cytotoxic T cells (or CTLs) are CD8+ T cells T helper cells are CD4+ T cells. T cell precursors include naïve T cells, stem cell memory T (T_{SCM}) cells and central memory T (T_{CM}) cells.

In some particular embodiments, the immune effector cells are cytolytic lymphocytes, more particularly cytolytic T cells. The immune cells may comprise CD8+ T cells or a mixture of CD4+ T cells and CD8+ T cells, for example comprising a 1:1 CD4+ to CD8+ T cell ratio. The T cells preferably contain a high percentage of less differentiated T cell subsets that have greater proliferative capacity, such as naïve T cells, stem cell memory T (T_{SCM}) cells and central memory T (T_{CM}) cells.

Immune cells are preferably obtained from the subject to be treated (autologous). However, in some embodiments, immune effector cell lines or donor effector cells (allogenic) may be used. Allogenic refers to histocompatible (HLA-compatible) cells.

As used herein, the term “individual”, “subject” or “patient” includes human and other mammalian subjects. Preferably, a patient, individual or subject according to the invention is a human.

Immune cells (including effector cells and/or precursors thereof) can be obtained from a number of sources such as blood or other body fluid and lymphoid tissue or organs (bone marrow, spleen, lymph node, cord blood, thymus) and others that are well-known in the art. In some embodiments, the immune cells are obtained from blood collected from a subject, in particular the subject to be treated.

Immune cells are isolated and cultured using standard methods that are well-known in the art. Immune cells may be isolated by leukapheresis, usually using differential centrifugation technique to separate leukocytes from whole blood. Specific sub-population of immune cells, in particular T cells, can be further isolated by positive or negative selection techniques, for example using flow cytometry assisted cell sorting or magnetic activated cell separation. For example, immune cells, in particular T cells, can be isolated using a combination of antibodies directed to surface markers unique to the positively selected cells, e.g., by incubation with antibody-conjugated beads for a time sufficient for positive selection of the desired immune cells. Alternatively, enrichment of immune cells, in particular T cells, can be performed by negative selection using a combination of antibodies directed to surface markers unique to the negatively selected cells. The isolated immune cells, in particular T cells, preferably contain a high percentage of less differentiated cell subsets that have greater proliferative capacity, such as naïve T cells, stem cell memory T (T_{SCM}) cells and central memory T (T_{CM}) cells.

The isolated immune cells, in particular T cells, are cultured in appropriate culture medium, for example supplemented with cytokines such as IL-7 and IL-15 for T cells, and optionally activated. Then, the isolated immune cells, in particular T cells are genetically modified with a CAR construct so as to express the CAR on their surface. The genetically modified (i.e., engineered) immune cells expressing the CAR may be expanded in appropriate culture medium, for example supplemented with cytokines such as IL-7 and IL-15 for T cells, before administration to the patient. The immune cells may be engineered in the presence of kinase inhibitors to generate CAR T cells with a less differentiated phenotype so as to improve engraftment, in vivo proliferation and effector activity of these cells.

The immune cells are genetically modified using standard nucleic acid delivery agents or systems suitable for the delivery and expression of nucleic acid into individual's immune effector cells, in particular suitable for gene therapy. The invention may use chemical, biological or physical delivery methods or a combination thereof. Such vectors that are well-known in the art include viral and non-viral vectors, wherein said vectors may be integrative or non-integrative; replicative or non-replicative.

The vector comprises the nucleic acid construct comprising the CAR in expressible form as described above. In order to assess the expression of a CAR polypeptide, the expression vector to be introduced into a cell can also contain either a selectable marker gene or a reporter gene or both to facilitate identification and selection of expressing cells from the population of cells sought to be transfected or infected with the expression vector. The selectable marker or reporter gene may be expressed from the same promoter as the CAR or from a different promoter. To express the CAR and the selectable marker or reporter gene from the same promoter, an Internal Ribosome Entry Site (IRES) or a viral 2A peptide (T2A) is inserted between the two coding sequences. The viral 2A peptide is a self-cleavable peptide. For example, the vector may further comprise CD19 or a detectable fragment thereof (e.g. detectable with CD19-specific antibodies).

Non-viral vector includes the various (non-viral) agents which are commonly used to either introduce or maintain nucleic acid into individual's cells. Agents which are used to introduce nucleic acid into individual's cells by various means include in particular polymer-based, particle-based, lipid-based, peptide-based delivery vehicles or combinations thereof, such as with no limitations cationic polymer, dendrimer, micelle, liposome, exosome, microparticle and nanoparticle including lipid nanoparticle (LNP); and cell penetrating peptides (CPP). CPP are in particular cationic peptides such as poly-L-Lysine (PLL), oligo-arginine, Tat peptides, Penetratin or Transportan peptides and derivatives thereof such as for example Pip. Agents which are used to maintain nucleic acid into individual's cells (either integrated into chromosome(s) or else in extrachromosomal form) include in particular naked nucleic acid vectors such as plasmids, transposons and mini-circles, and gene-editing and RNA-editing systems. Transposon includes in particular the hyperactive Sleeping Beauty (SB100X) transposon system (Mates et al. 2009). Gene-editing and RNA-editing systems may use any site-specific endonuclease such as Cas nuclease, TALEN, meganuclease, zinc finger nuclease and the like. Nucleic acid may also be introduced into individual's cells by physical means such as particle bombardment, microinjection, electroporation, and the like. In addition, these approaches can advantageously be combined to introduce and maintain the nucleic acid of the invention into individual's cells.

Viral vectors are by nature capable of penetrating into cells and delivering nucleic acid(s) of interest into cells, according to a process named as viral transduction. As used herein, the term “viral vector” refers to a non-replicating, non-pathogenic virus engineered for the delivery of genetic material into cells. In viral vectors, viral genes essential for replication and virulence are replaced with an expression cassette for the transgene of interest. Thus, the viral vector genome comprises the transgene expression cassette flanked by the viral sequences required for viral vector production. As used herein, the term “recombinant virus” refers to a virus, in particular a viral vector, produced by standard recombinant DNA technology techniques that are known in the art. As used herein, the term “virus particle” or “viral particle” is intended to mean the extracellular form of a non-pathogenic virus, in particular a viral vector, composed of genetic material made from either DNA or RNA surrounded by a protein coat, called the capsid, and in some cases an envelope derived from portions of host cell membranes and including viral glycoproteins. As used herein, a viral vector refers to a viral vector particle.

A first type of vector for delivering the nucleic acid (nucleic acid construct) of the invention is a viral vector, in particular suitable for gene therapy of individual's immune cells. In particular, the viral vector may be derived from a non-pathogenic parvovirus such as adeno-associated virus (AAV), a retrovirus such as a gammaretrovirus, spumavirus and lentivirus, and an adenovirus. The viral vector is preferably an integrating vector such as AAV or lentivirus vector, preferably lentivirus vector. The vector comprises the viral sequences required for viral vector production such as the lentiviral LTR sequences or the AAV ITR sequences flanking the expression cassette. Lentivirus vector may be pseudotyped with another envelope glycoprotein, preferably another viral envelope glycoprotein such as VSV-G. Pseudoptyped lentiviral vector improves the transduction of individual's immune cells. Lentiviral vector is advantageously a self-inactivating (SIN) lentiviral vector (3^{rd} generation) as originally described in Zufferey et al. (J. Virol., 1998, 72, 9873-9880). In some particular embodiments, the lentiviral vector is derived from the lentiviral transfer plasmid of SEQ ID NO: 19 or 20.

Another type of vector for delivering the nucleic acid (nucleic acid construct) of the invention is a particle or vesicle, macromolecule complexes, nanocapsules, microspheres, beads, and lipid-based systems including oil-in-water emulsions, micelles, mixed micelles, in particular lipid-based micro- or nano-vesicle or particle such as liposome or lipid nanoparticle (LNP). In more particular embodiments, the nucleic acid is RNA and the vector is a particle or vesicle as described above.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20 DC. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectaminenucleic acid complexes.

### Therapeutic Use

The engineered immune cell according to the present disclosure is used in Adoptive cell therapy (ACT) of skeletal muscle fibrosis associated with muscular dystrophies.

Adoptive cell therapy (ACT) also called adoptive T cell therapy, adoptive cell transfer, cellular adoptive immunotherapy or T-cell transfer therapy is a type of immunotherapy in which immune cells, in particular T cells, are engineered to recognize an antigen of interest for therapy (redirected T cell immunotherapy, CAR T cell therapy) administered to a patient to help the immune system fight diseases.

The immune cells used in adoptive cell therapy may be autologous or allogenic immune cells. Allogenic refers to histocompatible (HLA-compatible) cells. For example, a population of immune cells, in particular lymphocytes such as T cells, obtained from a subject sample is engineered to express a CAR specific for FAP according to the present disclosure and re-administered to the subject having muscular dystrophy-associated skeletal fibrosis. Alternatively, a population of allogenic immune cells, in particular T cells obtained from a donor sample is engineered to express a CAR specific for FAP according to the present disclosure and administered to a recipient subject having a muscular dystrophy-associated skeletal fibrosis.

The engineered immune cells may be delivered to the individual in need thereof by any appropriate mean such as for example by intravenous injection (infusion or perfusion), or injection in the tissue of interest (implantation).

Skeletal muscle fibrosis is a common hallmark of chronic skeletal muscle degenerative disorders, such as muscular dystrophies. Muscular dystrophies are a genetically and clinically heterogeneous group of neuromuscular diseases that cause progressive weakness and breakdown of skeletal muscles over time. Despite the variations in clinical manifestations, the dystrophies share a common histological appearance characterized by variation in muscle fiber size and myofiber necrosis. Muscular dystrophies are caused by spontaneous or inherited mutations in a variety of genes, involved in muscle development or function. Muscular dystrophies may be X-linked recessive, autosomal recessive or autosomal dominant.

Over 30 different disorders are classified as muscular dystrophies Of those, Duchenne muscular dystrophy (DMD) accounts for approximately 50% of cases and affects males beginning around the age of four. Other relatively common muscular dystrophies include Becker muscular dystrophy (BMD); facio-scapulo-humeral muscular dystrophy and myotonic dystrophy, whereas limb-girdle muscular dystrophy and congenital muscular dystrophy are themselves groups of several, usullay ultrarare, genetic disorders.
Dystrophinopathies are a spectrum of X-linked muscle diseases caused by pathogenic variants in DMD gene, which encodes the protein dystrophin. Dystrophinopathies comprises Duchenne muscular dystrophy (DMD) and Becker muscular dystrophy (BMD).
The Limb-girdle muscular dystrophies (LGMDs) are a group of disorders that are clinically similar to DMD but occur in both sexes as a result of autosomal recessive and autosomal dominant inheritance. Limb-girdle dystrophies are caused by mutation of genes that encode sarcoglycans and other proteins associated with the muscle cell membrane, which interact with dystrophin. The term LGMD1 refers to genetic types showing dominant inheritance (autosomal dominant), whereas LGMD2 refers to types with autosomal recessive inheritance. Pathogenic variants at more than 50 loci have been reported (LGMD1A to LGMD1G; LGMD2A to LGMD2W). Calpainopathy (LGMD2A) is caused by mutation of the gene CAPN3 with more than 450 pathogenic variants described. Contributing genes to LGMD phenotype include: anoctamin 5 (ANO5), blood vessel epicardial substance (BVES), calpain 3 (CAPN3), caveolin 3 (CAV3), CDP-L-ribitol pyrophosphorylase A (CRPPA), dystroglycan 1 (DAG1), desmin (DES), DnaJ heat shock protein family (Hsp40) homolog, subfamily B, member 6 (DNAJB), dysferlin (DYSF), fukutin related protein (FKRP), fukutin (FKTN), GDP-mannose pyrophosphorylase B (GMPPB), heterogeneous nuclear ribonucleoprotein D like (HNRNPDL), LIM zinc finger domain containing 2 (LIMS2), laminin A/C (LMNA), myotilin (MYOT), plectin (PLEC), protein O-glucosyltransferase 1 (PLOGLUT1), protein O-linked mannose N-acetylglucosaminyltransferase 1 (beta 1,2-) (POMGNT1), protein O-mannose kinase (POMK), protein O-mannosyltransferase 1 (POMT1), protein O-mannosyltransferase 2 (POMT2), sarcoglycan alpha (SGCA), sarcoglycan beta (SGCB), sarcoglycan delta (SGCD), sarcoglycan gamma (SGCG), titin-cap or telethonin (TCAP), transportin 3 (TNPO3), torsin 1A interacting protein (TORIAIPI), trafficking protein particle complex 11 (TRAPPC11), E3-ubiquitin ligase or tripartite motif containing 32 (TRIM 32) and titin (TTN). Major contributing genes to LGMD phenotype include CAPN3, DYSF, FKRP and ANO5 (Babi Ramesh Reddy Nallamilli et al., Annals of Clinical and Translational Neurology, 2018, 5, 1574-1587.
The Emery-Dreifuss Muscular Dystrophy (EDMD) caused by defects in one of the gene including the EMD gene (coding for emerin), the FHL1 gene and the LMNA gene (encoding lamin A and C).
Nesprin-1 and Nesprin-2 related muscular dystrophy caused by defects in the SYNE1 and SYNE2 gene, respectively; LUMA related muscular dystrophy caused by defects in the TMEM43 gene; LAP1B related muscular dystrophy caused by defects in the TORIAIPI gene.
Facio-scapulo-humeral muscular dystrophy, type 1 (FSHD1A), such as associated with defect in the DUX4 gene (contraction of the D4Z4 macrosatellite repeat in the subtelomeric region of chromosome 4q35) or the FRG1 gene; Facio-scapulo-humeral muscular dystrophy, type 2 (FSHD1B) caused by defects in the SMCHD1 gene.
Oculopharyngeal dystrophy (autosomal dominant) caused by defect in PABPN1 gene encoding Polyadenate binding protein nuclear 1.
Myotonic dystrophy (autosomal dominant) caused by defect in DMPK gene encoding DM1 protein kinase (Type 1) or CNBP encoding CCHC-type zinc finger nucleic acid binding protein (Type 2).
Congenital muscle dystrophies (CMDs) are a heterogeneous group of disorders characterized by muscle weakness from birth, or shortly after, and variable clinical manifestations of the eye and central nervous system. CMDs are autosomal recessively inherited, except in some cases. At least 35 genes are involved in CMDs with abnormalities at different cellular levels:
   the extracellular matrix
   MDC1A: LAMA2 gene; Collagen 6-related dystrophies (UCMD, BM): COL6A1, COL6A2, COL6A3 genes; Integrinopathies: ITGA7, ITGA9 genes; CMD with hyperlaxity: ITGA9, LAMR1, ACVR2B genes.
   the dystrophin-associated glycoprotein complex: alphadystroglycanopathies (DGP): DAG1, POMPT1, POMPT2, POMGNT1, POMGNT2 GTDC2, FKTN, FKRP, LARGE, ISPD, TMEM5, B3GALNT2; B4GAT1/B3GNT1, DPM1, DPM2, DPM3, POMK/SKG196, GMPPB, DOLK, DAG1 and INPP5K genes.
the endoplasmic reticulum: rigid spine syndrome [RSMD1]: SEPN1 gene
the nuclear envelope: LMNA-related CMD; [L-CMD]: LMNA gene; Nesprin-1-related CMD: SYNE1 gene,
ER to Golgi apparatus trafficking: CMD with fatty liver and infantile-onset cataract: TRAPCC11, GOSR2 genes.
Other CMDs: RYR1, CHKB, MSTO1, MICU1, INPP5K, SIL-1, MCOLN1, GGPS1, FHFL1, ALG13, ACTA1 genes.
The most common forms of CMDs are COL6-related diseases, dystroglycanopathies (DGP), laminin-alpha 2 (LAMA2) related dystrophies and selenoprotein N related myopathy (SEPN1).
Other genes: Polymerase I and transcript release factor (PTRF), Heterogeneous nuclear ribonucleoprotein D-like (HNRNPDL), Plectin (PLEC), Valosin-containing protein (VCP), LIM and senescent cell antigen-like domains 2 (LIMS2).

Engineered immune cells according to the present disclosure are used for treating skeletal fibrosis in muscular dystrophies, which means for treating skeletal muscle fibrosis associated with muscular dystrophies. Engineered immune cells according to the present disclosure are capable of immunologically targeting and depleting fibrotic cells in skeletal muscle of subjects suffering from muscular dystrophy. The engineered immune cells according to the present disclosure provides a treatment for muscular dystrophies, in particular in combination with gene therapy.

The administration of engineered immune cells according to the present disclosure reduces skeletal muscle fibrosis in subjects suffering from muscular dystrophy. Reduction of skeletal muscle fibrosis may be determined using standard methods that are well-known in the art and disclosed in the examples. These methods include in particular measuring expression levels of fibrosis biomarkers, in particular collagen type III (collagen type III alpha 1 chain; COL3A1 gene) or others; histological or immunohistological analysis of skeletal muscle samples; and a combination thereof. In some embodiments, the administration of engineered immune cells according to the present disclosure to subjects suffering from muscular dystrophy, reduces the levels of collagen type III (Col3) in skeletal muscles, compared to untreated controls.

In some embodiments, the engineered immune cells are used for the treatment of muscular dystrophies chosen from: Dystrophynopathies, Limb-girdle muscular dystrophies and and Congenital muscle dystrophies, in particular Dystrophynopathies associated with mutations in the DMD gene; Limb-girdle muscular dystrophies (LGMDs) associated with mutations in CAPN3, DYSF, FKRP, ANO5, DNAJB6, SGCA, SGCB or SGCG) genes, and Congenital muscle dystrophies associated with mutations in COL6A1, COL6A2, COL6A3, LAMA2 or SEPN1 genes; preferably Duchenne muscular dystrophy (DMD gene).

In some embodiments, the engineered immune cells, preferably in combination with gene therapy, are used for the treatment of skeletal muscle disease or damage in patients suffering from muscular dystrophies. This means that the immunotherapy with the engineered immune cells or immunotherapy combined with gene therapy according to the present disclosure, is used for the treatment of the skeletal muscle damage component of muscular dystrophies. The treatment is used in particular to improve skeletal muscle function and thereby ameliorate the symptoms associated with skeletal muscle disease such as muscle weakness, stiffness, difficulty in walking, climbing, running, rising from a sitting or lying position, frequent falls and others.

In some embodiments, the engineered immune cells are used for the treatment of the forms of muscular dystrophies that affect essentially skeletal muscles or that affect exclusively skeletal muscles (skeletal muscle dystrophies). In some embodiments, the engineered immune cells are used for the treatment of the forms of muscular dystrophies that do not affect cardiac muscle (heart) and/or are not associated with cardiac disease (cardiomyopathy). In some embodiments, the engineered immune cells are used for the treatment of muscular dystrophies in subjects who do not have a cardiomyopathy.

“Treatment”, or “treating” as used herein, is defined as the application or administration of a therapeutic agent or combination of therapeutic agents to a patient, or application or administration of said therapeutic agents to an isolated tissue or cell line from a patient, who has a disease with the purpose to cure, heal, alleviate, relieve, alter, remedy, ameliorate, improve or affect the disease, or any symptom of the disease. In particular, the terms “treat’ or treatment” refers to reducing or alleviating at least one adverse clinical symptom associated with the disease.

The term “treatment” or “treating” is also used herein in the context of administering the therapeutic agents prophylactically.

In the various embodiments of the present invention, the pharmaceutical composition comprises a therapeutically effective amount of engineered immune cell. In the context of the invention a therapeutically effective amount refers to a dose sufficient for reversing, alleviating or inhibiting the progress of the disorder or condition to which such term applies, or reversing, alleviating or inhibiting the progress of one or more symptoms of the disorder or condition to which such term applies. The term “effective dose” or “effective dosage” is defined as an amount sufficient to achieve, or at least partially achieve, the desired effect.

The effective dose is determined and adjusted depending on factors such as the composition used, the route of administration, the physical characteristics of the individual under consideration such as sex, age and weight, concurrent medication, and other factors, that those skilled in the medical arts will recognize. The effective dose can be determined by standard clinical techniques. In addition, in vivo and/or in vitro assays may optionally be employed to help predict optimal dosage ranges.

The precise amount of the compositions of the present invention to be administered can be determined by a physician with consideration of individual differences in age, weight, and condition of the patient (subject). It can generally be stated that a pharmaceutical composition comprising the T cells described herein may be administered at a dosage of 10⁴ to 10⁹ cells/kg body weight, preferably 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

The dosage of the above treatments to be administered to a patient will vary with the precise nature of the condition being treated and the recipient of the treatment. The scaling of dosages for human administration can be performed according to art-accepted practices. Strategies for CAR T cell dosing and scheduling have been discussed (Ertl et al, 2011, Cancer Res, 71:3175-81; Junghans, 2010, Journal of Translational Medicine, 8:55).

In certain embodiments, it may be desired to administer activated T cells to a subject and then subsequently redraw blood (or have an apheresis performed), activate T cells therefrom according to the present invention, and reinfuse the patient with these activated and expanded T cells. This process can be carried out multiple times every few weeks. In certain embodiments, T cells can be activated from blood draws of from 20 cc to 400 cc. In certain embodiments, T cells are activated from blood draws of 20 cc, 30 cc, 40 cc, 50 cc, 60 cc, 70 cc, 80 cc, 90 cc, or 100 cc. Not to be bound by theory, using this multiple blood draw/multiple reinfusion protocol may serve to select out certain populations of T cells.

In the various embodiments of the present invention, the pharmaceutical composition comprises a pharmaceutically acceptable carrier and/or vehicle.

A “pharmaceutically acceptable carrier” refers to a vehicle in which the therapeutic is administered and that does not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

Preferably, the pharmaceutical composition contains vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or suspensions. The solution or suspension may comprise additives which are compatible with viral vectors and do not prevent viral vector particle entry into target cells. In all cases, the form must be sterile and must be fluid to the extent that easy syringe ability exists. It must be stable under the conditions of manufacture and storage and must be preserved against the contaminating action of microorganisms, such as bacteria and fungi. An example of an appropriate solution is a buffer, such as phosphate buffered saline (PBS) or Ringer lactate.

The pharmaceutical composition may also comprise an additional therapeutic agent, in particular an agent useful for the treatment of a disease according to the present disclosure.

The pharmaceutical composition of the present invention is generally administered according to known procedures, at dosages and for periods of time effective to induce a therapeutic effect in the patient. The pharmaceutical composition may be administered by any convenient route, such as in a non-limiting manner by injection, perfusion or implantation. The administration can be systemic, local or systemic combined with local. Systemic administration is preferably intravascular such as intravenous (IV) or intraarterial; intraperitoneal (IP); intradermal (ID), epidural or else. In some preferred embodiments, the administration is parenteral, preferably intravascular such as intravenous (IV) or intraarterial. The parenteral administration is advantageously by injection or perfusion.

The engineered immune cell or pharmaceutical composition described herein may be used in combination with other biologically active agents, wherein the combined use is by simultaneous, separate or sequential administration.

In some embodiments, immunotherapy according to the invention is combined with gene therapy of muscular dystrophies. Gene therapy is advantageously performed with recombinant AAV (rAAV) vectors, in particular those disclosed in WO2019/193119, WO2020/216861, WO2021/219762 WO2022/003211, WO2022/053630. The recombinant AAV vector comprises a transgene intended for use in gene replacement or gene editing of a mutated gene associated with the muscular dystrophy to be treated in the subject. In some particular embodiments, immunotherapy according to the invention is combined with gene therapy of muscular dystrophies chosen from: Dystrophynopathies, Limb-girdle muscular dystrophies and Congenital muscle dystrophies; more particularly Dystrophynopathies (DMD gene), Limb-girdle muscular dystrophies (LGMDs) (CAPN3, DYSF, FKRP, ANO5, DNAJB6 SGCA, SGCB, or SGCG genes), and Congenital muscle dystrophies (COL6A1, COL6A2, COL6A3, LAMA2, or SEPN1 genes); preferably Dystrophynopathies (DMD gene). In some more particular embodiments, the combination therapy comprises the administration of a reduced dose of vector for gene therapy compared to the use of the vector for gene therapy without the immune cell. The possibility of lowering the dose of gene therapy vector to be administered, potentially implies greater functional efficacy and less risk of toxicity. In some particular embodiments, gene therapy of the disease is performed after immunotherapy according to the present invention.

The invention provides a pharmaceutical combination for use in the treatment of skeletal fibrosis in a subject in need thereof, comprising therapeutically effective amounts of:
(i) an engineered immune cell according to the present disclosure; and
(ii) a gene therapy vector, preferably recombinant AAV vector according to the present disclosure.

In some embodiments, the pharmaceutical combination is for sequential use, wherein the treatment comprises at least one initial administration of the engineered immune cell before the administration of the gene therapy vector, preferably rAAV vector.

The invention provides a method for treating skeletal fibrosis in muscular dystrophies as described above, comprising: administering to a patient in need thereof a therapeutically effective amount of the pharmaceutical composition as described above; preferably further comprising the administration of a therapeutically effective amount of gene therapy vector, in particular rAAV vector, according to the present disclosure.

A further aspect of the invention relates to the use of an engineered immune cell according to the present disclosure in the manufacture of a medicament for the treatment of skeletal fibrosis in muscular dystrophies according to the present disclosure.

Another aspect of the invention relates to the use of an engineered immune cell according to the present disclosure for the treatment of skeletal fibrosis in muscular dystrophies according to the present disclosure; preferably in combination with a gene therapy vector, more preferably rAAV vector, according to the present disclosure.

A further aspect of the invention relates to a pharmaceutical composition for treatment of skeletal fibrosis in muscular dystrophies according to the present disclosure, comprising an engineered immune cell according to the present disclosure as an active component.

The invention also relates to the use of the engineered immune cells according to the disclosure in therapies that reduce fibrosis to enhance AAV gene transfer efficacy.

The various embodiments of the present disclosure can be combined with each other and the present disclosure encompasses the various combinations of embodiments of the present disclosure.

The practice of the present invention will employ, unless otherwise indicated, conventional techniques, which are within the skill of the art. Such techniques are explained fully in the literature.

The invention will now be exemplified with the following examples, which are not limitative, with reference to the attached drawings in which:

### FIGURE LEGENDS

FIG. 1: Schematic representation of the third-generation lentiviral vectors (A) pCCL-EF1a-scFvFAP-CD28-4.1BB-CD3ζ-T2A-ΔCD19-WPRE and (B) pCCL-EF1a-scFvFAP-CD28-4.1BB-CD3ζ-WPRE where scFv against mFAP is fused to T-cell signaling domains CD28, 4.1BB and CD3ζ. The selection marker ΔCD19 is also indicated (A only).

FIG. 2: Phenotypic multi-color flow cytometric analysis of T lymphocytes (LT) non-transduced (left panels) or transduced (right panels) after 7 days of activation including 5 days of transduction (PT). Cells have been immuno-labelled prior analysis with a mouse anti-CD3, -CD4 and -CD8, and a human anti-CD19 and analysis is performed on live-gated cells.

FIG. 3: Murine FAP expression in 3T3 and 3T3-FAP cells. (A) mRNA FAP expression measured by ddPCR in 3T3, 3T3-FAP and a mixed (50:50) cells population of 3T3 and 3T3-FAP cells. Absence of FAP expression is confirmed in 3T3 and is specifically expressed in the stable cell line 3T3-FAP. (B) Murine FAP expression revealed by immunocytochemistry using a murine anti-FAP antibody.

FIG. 4: Luciferase assay after 24 h coculture between 3T3-Luc2 or 3T3-FAP-Luc2 and T-cells or FAP-CAR-T following ratio 1:7.5 and 1:15 (1 3T3 for 7.5 or 15 T). These results correspond to two experiments done in triplicates (one dot corresponds to one value). p values were calculated with Student's T-test

FIG. 5: Degranulation assay after 6 h coculture between 3T3-Luc2 or 3T3-FAP-Luc2 and T-cells or FAP-CAR-T following the ratio 1:15.

FIG. 6: Kinetic of FAP (top) and Col3 (bottom) expression relative to P0 in the Tibialis (TA), Gastrocnemius (GA) and Extensor Digitorum Longus (EDL) from DBA2 and DBA2-MDX mice aged from 2 to 4 month-old. Statistical analysis: unpaired Student's T-test.

FIG. 7: Schematic representation of the FAP-CAR-T treatment and details for each study. Two to three month-old DBA2-MDX mice receive 2 consecutive doses of FAP-CAR-T cells (low or high) and skeletal muscles were collected 2 weeks after the second infusion for molecular and histological analyses. The number (Nb) of T-cells injected represents the total number of cells in the preparation of CAR-T cells. The Input is calculated from the vector copy number (VCN) in the preparation of CAR-T cells and which represents the active principle. These Input numbers vary depending on the separate preparations of CAR-T cells which were made and are reported in the table for each injection.

FIG. 8: mRNA expression of FAP (top) and Col3 (bottom) in the Tibialis (TA), Extensor Digitorum Longus (EDL) and heart from DBA2 (WT) and DBA2-MDX mice controls (white bars) or treated with LT (darker grey bars) or FAP-CAR-T cells at r high doses (1×10⁶ cells light grey bars) (n=6 mice per group). Statistical analysis: Mann and Whitney unpaired t test.

FIG. 9: Sirius Red histological sections of TA, EDL and Heart from DBA2 mice, untreated DBA2-MDX controls and treated DBA2-MDX mice. The dark intensity staining represents collagen fibers (Obf: 10×) (A). Collagen deposit were quantified on muscle section (B).

FIG. 10: Schematic representation of the combined FAP-CAR-T treatment and AAV-microdystrophin gene therapy in MDX mice model of DMD.

FIG. 11: Increase of microdystrophin mRNA expression in TA and EDL after FAP-CAR-T treatment. MDX mice were treated as shown on FIG. 10. MDX mice treated only with a 10-fold higher dose of AAV-MD1 (4×10¹³ vg/kg) are used as positive control. Microdystrophin mRNA levels were determined by digital droplet PCR using primers from Table 4 and normalized to levels of murine Mpz (myelin protein zero or PO) mRNA.

FIG. 12: Increase of AAV viral genome copies in TA and EDL after FAP-CAR-T treatment. MDX mice were treated as shown on FIG. 10. MDX mice treated only with a 10-fold higher dose of AAV-MD1 (4×10¹³ vg/kg) are used as positive control. Viral genome copies was determined by digital droplet PCR using primers from Table 4.

FIG. 13: Increase of microdystrophin positive fibers in TA and EDL after FAP-CAR-T treatment. MDX mice were treated as shown on FIG. 10. MDX mice treated only with a 10-fold higher dose of AAV-MD1 (4×10¹³ vg/kg) are used as positive control. Microdystrophin positive fibers were quantified after immunostaining on muscle sections.

FIG. 14: Increase of microdystrophin protein expression in TA and EDL after FAP-CAR-T treatment. MDX mice were treated as shown on FIG. 10. MDX mice treated only with a 10-fold higher dose of AAV-MD1 (4×10¹³ vg/kg) are used as positive control. Microdystrophin protein expression was quantified with simple western blot.

FIG. 15: Decrease of collagen deposit area in TA and EDL after FAP-CAR-T treatment. MDX mice were treated as shown on FIG. 10. MDX mice treated only with a 10-fold higher dose of AAV-MD1 (4×10¹³ vg/kg) are used as positive control. Collagen deposit were quantified on muscle section.

### EXAMPLES

### Materials and Methods

### 1. Plasmid Construction

1.1 Construction of pCCL-EF1a-scFvFAP-CD28-4.1BB-CD35-T2A-4CD19-WPRE

From the construct, pCCL-EF1a-scFvCD123-CD28-4.1BB-CD3ζ-T2A-ΔCD19-WPRE previously described in Bole-Richard et al., Leukemia, 2020, 34, 3228-3241, the fragment scFvCD123 was removed using the restriction enzymes PspXI and BstEII generating an acceptor backbone for the scFv FAP fragment. The fragment scFv FAP was synthetized from the sequence of an anti-murine FAP monoclonal antibody described as SEQ ID NO: 3 in the international application WO 2014/055442 and listed below as SEQ ID NO: 18, adding at each extremity the restriction enzyme sequences PspXI and BstEII. The scFvFAP fragment was digested with the restriction enzymes PspXI and BstEII, ligated into the PspXI and BstEII-digested acceptor pCCL backbone and the product was used to transform competent XL10 cells. Colonies were screened by digestion and the correct clones were confirmed by sequencing. pCCL-EF1a-scFvCD123-CD28-4.1BB-CD32-T2A-ΔCD19-WPRE corresponds to the nucleotide sequence SEQ ID NO: 19.

1.2 Construction of pCCL-EF1a-scFvFAP-CD28-4.1BB-CD3ζ-WPRE

From the pCCL-EF1a-scFvFAP-CD28-4.1BB-CD32-T2A-ΔCD19-WPRE plasmid (a) a PCR reaction was done in order to generate the fragment EcoR1-scFv-FAP-T2A-Sal1-EcoR1, (b) the fragment scfvFAP-CD28-4.1BB-CD32-T2A-ΔCD19 was removed using the restriction enzyme EcoRI and (c) the PCR fragment generated in (a) that does not contain ΔCD19 anymore was ligated to the EcoR1-digested pCCL backbone obtained in (b) to generate the pCCL-EF1a-scfvFAP-CD28-4.1BB-CD32-WPRE construct (SEQ ID NO: 20). Such product was used to transform competent XL10 cells. Colonies were screened by digestion and correct clones were confirmed by sequencing.

1.3 Construction of pCCL-SFFV-mFAP-WPRE

From the construct pCCL-SFFV-Luc2-WPRE available in the laboratory, the Luciferase 2 fragment was removed using the restriction enzymes BamHI and SalI. A plasmid containing the sequence coding for the mouse FAP protein was purchased from R&D Systems (RDC2905) and the mFAP cDNA fragment was extracted using the restriction enzymes BamHI and SalI prior ligation into the PCCL backbone in order to obtain pCCL-SFFV-mFAP-WPRE. The new product was used to transform competent XL10 cells. Colonies were screened by digestion and correct clones were confirmed by sequencing.

### 2. Lentiviral Production and Generation of Stably-Transduced Target and Control Cell Lines

Lentiviral vectors were produced by transient transfection of HEK293T cells using calcium phosphate and 4 plasmids including a transfer plasmid and three accessory plasmids (HIV-1 gagpol, HIV-Rev, and VSV-G). The following transfer plasmids were used: pCCL-EF1a-scFvFAP-CD28-4.1BB-CD32-T2A-ΔCD19-WPRE, pCCL-EF1a-scFvFAP-CD28-4.1BB-CD32-WPRE, pCCL-SFFV-mFAP-WPRE and pCCL-SFFV-Luc2-WPRE. The harvested particles were concentrated about 500 fold by ultracentrifugation (50,000×g, 2 h, 12° C.), suspended in phosphate buffered saline and cryopreserved at −80° C. The infectious titer of the vector was determined as infectious genome (IG)/mL titers on HCT116 cells using the ddPCR provirus primers normalized to albumin (see primers and probes sequences below):

TABLE 1
Oligonucleotide sequences

| Oligo | Sequence 5′→3′ |
|---|---|
| Alb.fw | GCTGTCATCTCTTGTGGGCTGT (SEQ ID NO: 21) |
| Alb.rv | ACTCATGGGAGCTGCTGGTTC (SEQ ID NO: 22) |
| Alb.pr | CGCACGGCAAGAGGCGAGG (SEQ ID NO: 23) |
| PRO.fw | CACTCCCAACGAAGACAAGA (SEQ ID NO: 24) |
| PRO.rv | TCTGGTTTCCCTTTCGCTTT (SEQ ID NO: 25) |
| PRO.pr | TCTCTAGCAGTGGCGCCCGAACAGG (SEQ ID |
| | NO: 26) |

The following stable cell lines expressing FAP, Luc2 or both transgenes: 3T3-FAP, 3T3-Luc2, 3T3-FAP-Luc2 were generated by lentiviral transduction in 1 ml complete DMEM media (1% glutamine, 1% Pen/Strep, 10% FBS) of the NIH-3T3 fibroblastic cell line using the vectors described above (1×10⁵ cells, 2×10⁶ IG/mL). Expression of the transgenes were confirmed by immunochemistry or bioluminescence assays.

### 3. FAP-CAR-T Cells Preparation

DBA2 primary murine splenic T cells were isolated using the “Pan T cell Negative Selection” kit as suggested by the manufacturer (Miltenyi Biotec), and transduced with the indicated LV. The cells (1×10⁶ cells/well) were incubated in 1 mL of complete medium in 24-well plates (complete medium=RPMI-1640+1% glutamine+1% Pen/Strep+10% FBS+50 uM Beta-mercaptoethanol+50 μg/mL IL-2+CD3/CD28 activation beads at a ratio of 1:1 (Gibco) prepared freshly). After 48 hours, cells (1×10⁶ cells/well in 1 mL complete medium) were mixed with FAP-CAR-T lentivirus (2×10⁷ IG/mL) and lentiboost (0.5 mg/mL) in a 24-well plate. After overnight incubation, cells were expanded in complete media for another 4 days. T-cells transduction efficacy was either determined by flow cytometry using an anti-human CD19 when using the LV pCCL-EF1a-scFvFAP-CD28-4.1BB-CD3ζ-T2A-ΔCD19-WPRE (see below) or by ddPCR to determine the vector copy number per cell in the case of the LV pCCL-EF1a-scFvFAP-CD28-4.1BB-CD3ζ-WPRE (using provirus ddPCR primers referenced to murine titin).

TABLE 2
Oligonucleotide sequences

| | |
|---|---|
| mTitin-F | 5′-AAAACGAGCAGTGACGTGAGC-3′ |
| | (SEQ ID NO: 27) |
| mTitin-R | 5′-TTCAGTCATGCTGCTAGCGC-3′ |
| | (SEQ ID NO: 28) |
| mTitin-P | 5′-TGCACGGAAGCGTCTCGTCTCAGTC-3′ |
| | (SEQ ID NO: 29) |

### 4. FAP-CAR-T Cells Phenotyping

After 7 days of culture, LT and FAP-CAR-T cells were incubated for 1 h with the following conjugated antibodies prior to FACS analysis: anti mouse-CD3, anti-mouse CD8 and anti-mouse CD4 and with the anti-human CD19, when using the LV pCCL-EF1a-scFvFAP-CD28-4.1BB-CD3ζ-T2A-ΔCD19-WPRE. After washing cells and adding the viability marker 7AAD, cells were analyzed by flow cytometry.

### 5. Luciferase Assay for FAP-CAR-T Cell Recent Cytotoxic Activity

5000 3T3-Luc2 control cells and 3T3-FAP-Luc2 target cells were plated in a 96 well plate (ViewPlate, Perkin Elmer, 6005181) in 100 μL of complete DMEM media (1% glutamine, 1% Pen/Strep, 10% FBS). The following day, FAP-CAR-T cells or control non-specific T lymphocytes (TL) were added at 2 different concentrations in 100 μL of medium containing 37,500 or 75,000 cells, to cuo-culture cells and targets for 24h. At the end of the co-culture, 100 μL of supernatant medium were removed from each well and mixed with 100 μL of luciferin solution (Bright-Glo Luciferase assay, Promega) to measure bioluminescence levels extemporaneously using a luminometer (560 nm).

### 6. Degranulation Assay to Measure FAP-Specific Cytotoxicity

Functional cytotoxic activity of FAP-CAR T cells was measured by the detection of CD107 lysosomal-associated membrane protein (LAMP) on the surface of recently degranulated cells. 5000 control 3T3 cells and 3T3-FAP target cells were plated in a 96 well plate in 100 μl of complete DMEM medium. The following day, 100 μL1 containing 75,000 FAP-CAR-T cells or control non-specific T lymphocytes (TL) were added to the corresponding wells together with brefeldin-A (1/2000) and 20 μL/mL of anti-CD107a. After 6 h incubation at 37° C., cells were washed with PBS-1× and stained with anti-CD8 antibody. After adding the viability marker 7AAD, cells were analyzed by flow cytometry.

### 7. Mice

For this entire study, only males were used. The DBA2 controls were purchased directly from Charles River Laboratories (DBA/2J, Ref: 625) while the DBA2/MDX are bred and obtained from the Centre d'Exploration et de Recherche Fonctionnelle Expérimentale (CERFE; Evry, France). Originally, the DBA2-MDX have been purchased from Jackson Laboratories (D2.B10-Dmd^{mdx}/J, Strain #013141) and they have been at CERFE for about 8-10 generations.

### 8. Expression of Fibrosis Marker Genes

The levels of murine collagen 3a1 and murine FAP gene mRNAs was measured by ddPCR in different muscle tissues and normalized to levels of murine Mpz (myelin protein zero or PO) mRNA. Tibialis anterior (TA), gastrocnemius (GA), extensor digitorum longus (EDL) and heart, were isolated from euthanized DBA2 and DBA2-MDX and directly conserved in RNAlater. RNA was extracted from these tissues using the RNeasy fibrous tissue kit (Qiagen) following manufacturer's recommendations. RNA was then reverse transcribed using the Verso cDNA Synthesis kit (ThermoFisher Scientific). For the ddPCR, 1× of ddPCR Supermix for Probes no dUTP (Biorad), 16 ng of complementary DNA and the following primers sets were used: ddPCR Gene Expression Assay: Fap, Mouse (Biorad, 10031252), ddPCR Gene Expression Assay: Col3a1, Mouse (Biorad, 10031252) and ddPCR Gene Expression Assay: Mpz, Mouse (Biorad, 10031255).

TABLE 3
Amplicon sequences

| Gene | Coordinates | Sequence of the amplicon |
|---|---|---|
| Mpz | mm10\|chr1: | ATCAAAAGGGCTGGGGGAGTCTCGCAAGGATAAG |
| | 171160004- | AAATAGCGGTTAGCGGGCCGGGGGGGGGTCGGG |
| | 171160125:+ | GGTCTGCGACGGAGTCCTCCAAAGGCTCTCAGGT |
| | | GGTGGTCATCGAGATGGAGC (SEQ ID NO: 30) |
| Fap | mm10\|chr2: | GAGCTCTCTTTGTGTTTCCTTCAGGTTTGTAAACTC |
| | 62556167- | TTGAGGGACGTAAGACAATGCATATCACCACTAA |
| | 62573849:+ | AGCAAGCGCAGCCAGGGTGGTAACTCCAAAGACA |
| | | GTTTTCAGCCATGTCTTC (SEQ ID NO: 31) |
| Col3a1 | mm10\|chr1: | GTGGAACCTGGTTTCTTCTCACCCTTCTTCATCCCA |
| | 45311767- | CTCTTATTTTGGCACAGCAGTCCAACGTAGATGAA |
| | 45321568:+ | TTGGGATGCAGCCACCTTGGTCAGTCCTATGAGTC |
| | | TAGAGATGTCTGGAAGCCAGAACCATGTCAAA |
| | | (SEQ ID NO: 32) |

After running the PCR [95° C. for 10 min+40×(94° C. for 30 sec, 60° C. for 1 min)+98° C. for 10 min], droplets are generated using the droplet generator QX200 (Biorad) and reactions are read and analyzed using the QuantaLife software (Biorad).

### 9. Mice and In Vivo Studies

Two to three month old anesthetized DBA2-MDX mice were injected intravenously into retro-orbital vein with either 5×10⁵ (Low dose) or 1×10⁶ (high dose) of control LT or specific FAP-CAR-T cells resuspended in 100 μl PBS-1×. Mice were reinjected one week later with the same dose and same cells. After two weeks, age-matched control mice (DBA2) and treated mice were euthanized and skeletal muscles collected for molecular and histological analyses.

For subsequent gene therapy, two to three month old DBA2-MDX mice were injected with two consecutive doses (1×10⁶) of FAP-CAR-T cells, and after two weeks of treatment the mice were injected with one dose (5·10¹² vg/kg) of rAAV9-microdystrophin (MD1) vector. For concomitant gene therapy, DBA2-MDX mice were injected with a first dose (1×10⁶) of FAP-CAR-T cells, and after two weeks of treatment the mice were injected concomitantly with a second dose (1×10⁶) of FAP-CAR-T cells and one dose (5·10¹² vg/kg) of rAAV9-microdystrophin (MD1) vector. Control DBA2-MDX mice were injected with two consecutive doses (1×10⁶) of LT, and two weeks later with one dose (5·10¹² vg/kg) of rAAV9-microdystrophin (MD1) vector (Bourg et al., Int. J. Mol. Sci., 2022, 23, 2016; doi: 10.3390/ijms23042016). After 6 weeks, blood was collected, and force tests (escape test an dTA in situ) were performed on control and treated mice (DBA2-MDX). After five weeks, control and treated mice (DBA2-MDX) were euthanized and heart and skeletal muscles (TA, ED) were collected for molecular and histological analyses.

### 10. Histological Analysis

TA, GA and EDL collected from study mice were immediately snap frozen in liquid nitrogen. Cryopreserved muscles were then sectioned (8 μm) using a cryostat and stained with the Sirius Red dye using standard procedures. Images were then acquired using the Axioscan microscope (10× Obj, Zeiss).

### Quantification of Microdystrophin Positive Fibers

Fiber cytoplasmic region enclosed within the membrane staining (Laminin) are segmented by morphological segmentation after contrast enhancement and artefact filtering (FiJi software 2.0.0-rc/1.52 p, Morpholib plugin v 1.4.1). Fibers are enlarged according to the magnification to capture the membrane region. Nuclei are detected from the DAPI channel using the local maxima detection. The fluorescence intensity in each object (fibers, fibers membrane and nuclei) is measured for each channel together with fibers shape and size.

Nuclei are associated to their parent fiber using the R software and all fluorescence and shape datas aggregated together. Non-fiber objects are filtered-out based on shape, size and fluorescence criteria. Positive fibers for any channel (Laminin, microdystrophin and nuclei) are detected based on the fluorescence distribution of negative control slices or slices from known negative condition and the percentage of microdystrophin fibers were determined

### Collagen Quantification on Muscle Section

Collagen deposits on muscle section are quantified using an open-source software for bioimage analysis: Qupath Software. For that, we created two Pixel classifiers to train the software on 3 muscle sections to identify representative region (collagen region) by surrounding them. The first one is for delimiting the tissue to be analysed and the second one is for identifying the region of collagen deposits. We quantified the surface area occupied by the collagen in relation with the total muscle section surface area.

### 11. Molecular Analysis

Microdystrophin mRNA expression levels and AAV vector copy number in muscle of DBA2-MDX mice receiving combined therapy were analysed by digital droplet PCR on cDNA or gDNA using the oligonucleotide primers and probes listed below.

TABLE 4
Oligonucleotide sequences

| Target gene | Name | Sequence |
|---|---|---|
| ITRG | AAV22mers.F | CTCCATCACTAGGGGTTCCTTG |
| | | (SEQ ID NO: 33) |
| ITRG | AAV18mers.R | GTAGATAAGTAGCATGGC |
| | | (SEQ ID NO: 34) |
| ITRG | AAV_MGB.P | TAGTTAATGATTAACCC |
| | | (SEQ ID NO: 35) |
| Microdystrophin | μdys.R | GGTTGTGCTGGTCCAGGGCGT |
| | | (SEQ ID NO: 36) |
| Microdystrophin | μdys.F | CCAACAAAGTGCCCTACTACATC |
| | | (SEQ ID NO: 37) |
| Microdystrophin | μdys.P | CCGAGCTGTACCAGAGCCTGGCC |
| | | (SEQ ID NO: 38) |
| Murine Mpz | MH181P0.F | CTCCAAGCAGATGCAGCAGA |
| (PO) | | (SEQ ID NO: 39) |
| Murine Mpz | M267PO.R | ATAGCCTTGCGCATCATGGT |
| (PO) | | (SEQ ID NO: 40) |
| Murine Mpz | M225PO.P | CCGTGGTGCTGATGGGCAAGAA |
| (PO) | | (SEQ ID NO: 41) |
| Murine Titin | mTitin-F | AAAACGAGCAGTGACGTGAGC |
| | | (SEQ ID NO: 27) |
| Murine Titin | mTitin-R | TTCAGTCATGCTGCTAGCGC |
| | | (SEQ ID NO: 28) |
| Murine Titin | mTitin-P | TGCACGGAAGCGTCTCGTCTCAGTC |
| | | (SEQ ID NO: 29) |

Quantification of Microdystrophin Expression with Simple Western Blot

Muscle proteins were extracted in RIPA buffer supplemented with Protease Inhibitor Cocktail EDTA-free (Roche) and Benzonase by homogenization. Total proteins were then quantified by BCA method, thanks to the Pierce BCA protein assay kit (Invitrogen) accordingly to manufacturer's instructions. Protein detection has been performed by capillary western blot, thanks to the JESS protein simple (Bio-techne), accordingly to manufacturer's directions. Micro-dystrophin detection has been performed by the antibody DysB (NCL-DYSB, Leica, 1:20) and its expression has been quantified by compass software.

### 12. Statistical Analyses

Differences between two groups were compared using Student's T test. Differences between multiple groups were compared with one-way analysis of variance (ANOVA). The statistics were calculated using the GraphPad Prism 7 software.

### Results

1. Construction of Lentiviral Gene Transfer Plasmids to Produce mFAP-Directed CAR T Cells

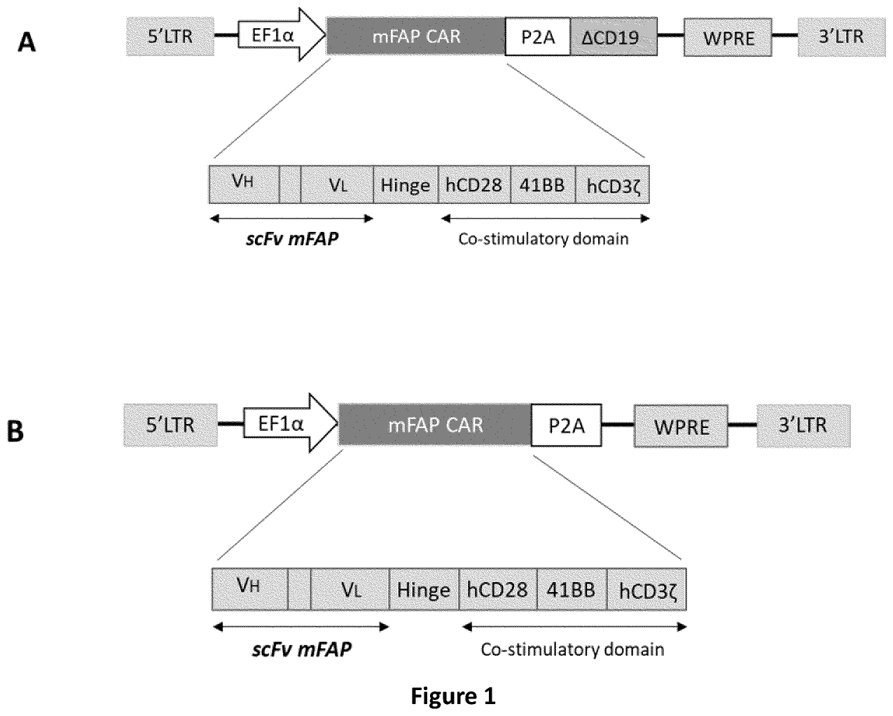

Advanced-generation HIV-1-derived lentiviral gene transfer plasmids were generated to express third-generation chimeric antigen receptor (CAR) constructs directed against murine fibroblast activation protein (FAP), containing the human CD28, 4.1BB and CD3zeta chain modules for co-stimulation, survival and signal transduction, eventually combined to a truncated human CD19 marker (FIG. 1A) or not (FIG. 1B). These constructs were obtained by replacing the single-chain variable Fragment (ScFv) immunoglobulin fusion protein sequence of another CAR previously described in Bôle-Richard et al., Leukemia, 2020, 34, 3228-3241, by a scFv specific for mFAP which was synthesized from the sequence published in the International application WO 2014/055442. Both lentiviral plasmids shown in FIG. 1 have been used to produce lentiviral vector for this study. The vectors were pseudotyped with VSVG and titered according to standard techniques present in the laboratory (Corre et al., Gene Therapy, 2022, 29, 536-543).

### 2. Production and Characterization of Murine FAP-CAR-T Cells

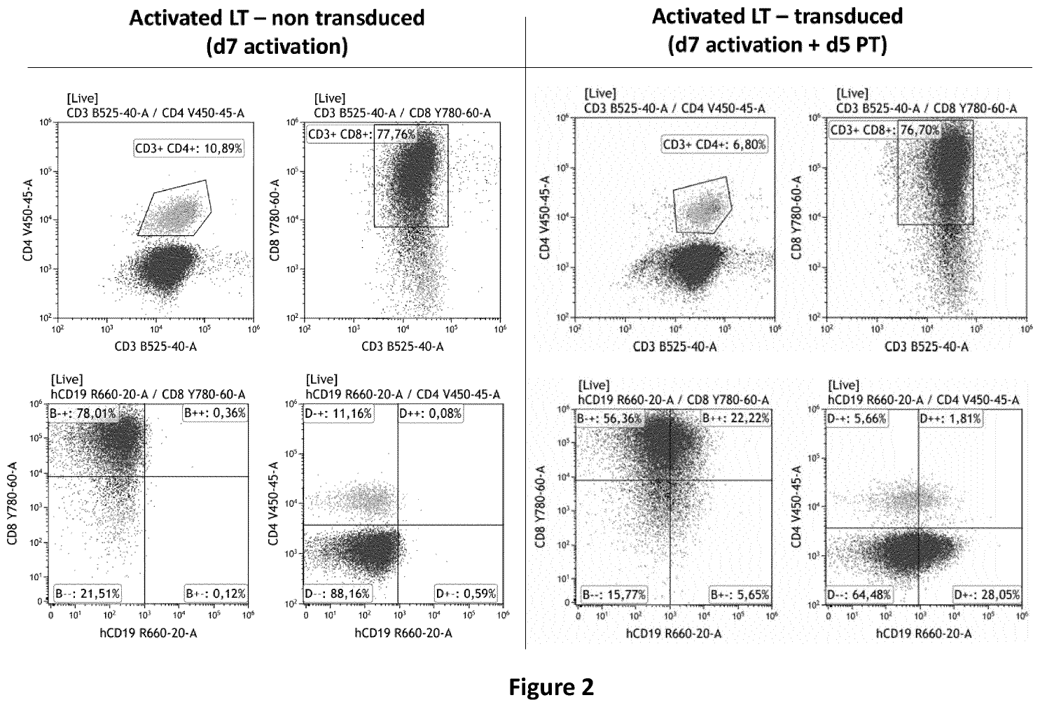

T lymphocytes (LT) isolated from spleens of 2 to 3 month-old DBA2 mice, were cultured, activated by CD28/CD3 beads and Interleukin-2 for 48h prior to lentiviral transduction. Five days post-transduction, the cell surface phenotype of the cells was characterized by flow cytometry (FIG. 2). Results show that after 7 days of activation, cells are mainly CD8+ and it can be extrapolated that ˜30% of cells are effectively transduced (CD19+).

### 3. FAP-CAR T Cell Generation and Functional Characterization In Vitro

### 3.1 Generation of Cells Expressing Mouse FAP

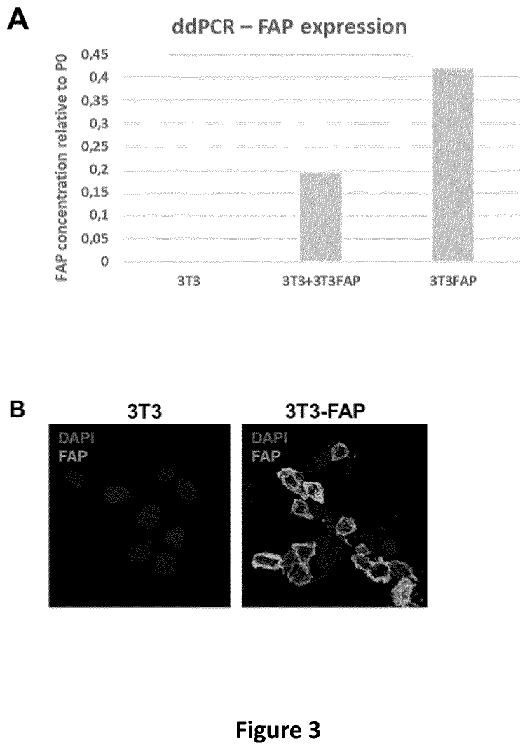

In order to test the specificity and efficacy of FAP-CAR-T in vitro, the murine fibroblastic cell line NIH-3T3 cells known to lack the mouse FAP, was used as negative control and to generate specific target cells. A lentiviral construct encoding the murine FAP was produced and used to transduce NIH-3T3 cells to generate the stable cell line 3T3-FAP which expresses high levels of the mFAP mRNA (FIG. 3-A) and of FAP protein (FIG. 3-B).

### 3.2 Generation of Cells for the Cytotoxicity Assay and Validation of FAP-CAR-T Cell Specific Cytotoxic Activity

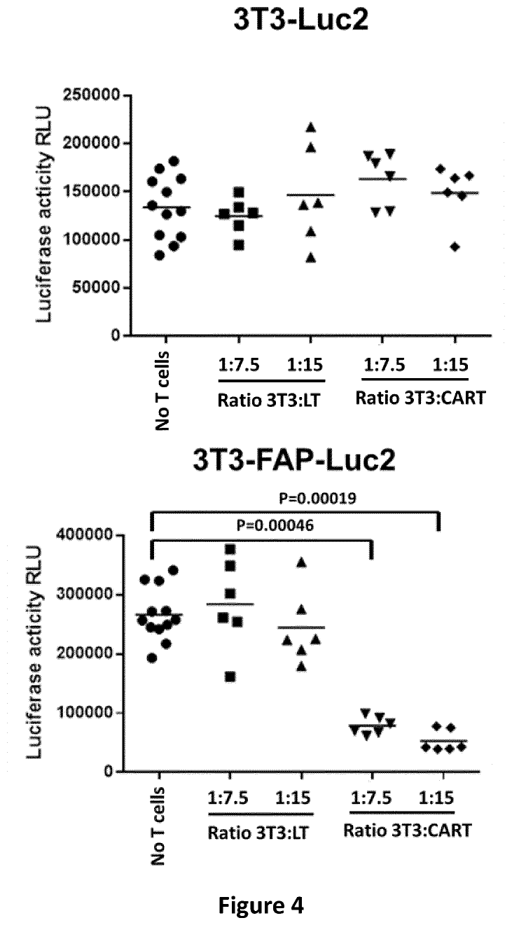

In order to measure FAP-CAR-T cell specific killing of FAP-bearing cells in vitro, two new stable cell lines were generated using lentiviral vectors coding for the bioluminescent marker Luciferin 2: 3T3-Luc2 and 3T3-FAP-Luc2 (FIG. 4). Results show that cell mortality (indicated by a decreased bioluminescence relative to controls) only occurs in a statistically significant manner when FAP-CAR-T cells are cocultured with cells expressing the FAP target.

### 3.3 Degranulation Assay

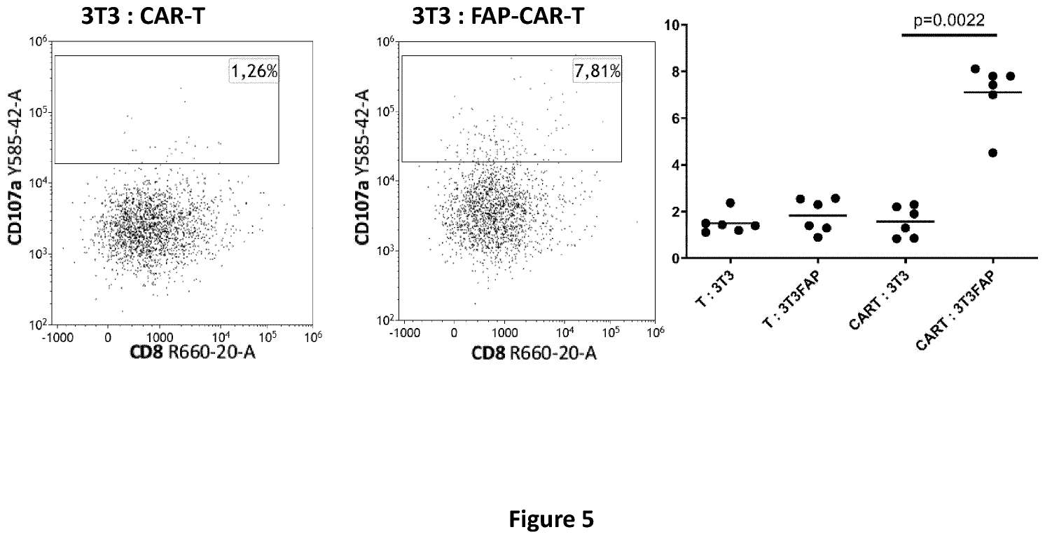

The functional properties of FAP-CAR-T cells were also tested in vitro using CD107a degranulation assay after coculture between 3T3 or 3T3-FAP and T-cells or FAP-CAR-T (FIG. 5). Results show that the FAP-CAR-T cells only degranulate when they are in presence of the murine FAP target.

### 4. Skeletal Muscle Fibrosis in the DBA2-MDX Model of Duchenne Muscular Dystrophy

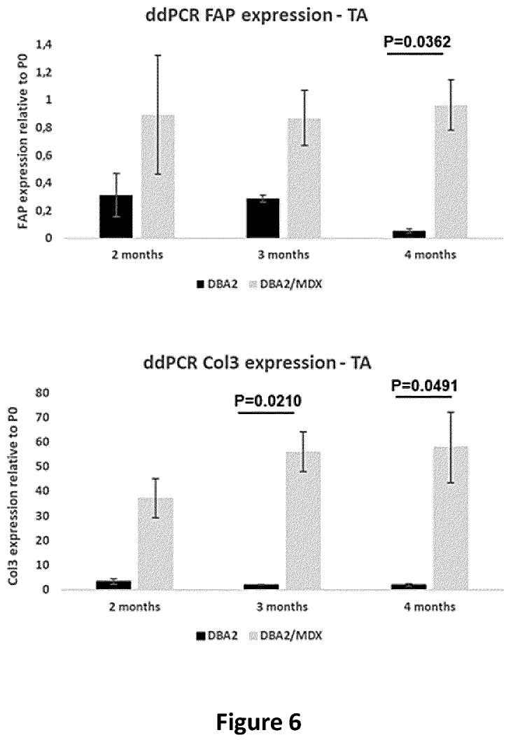

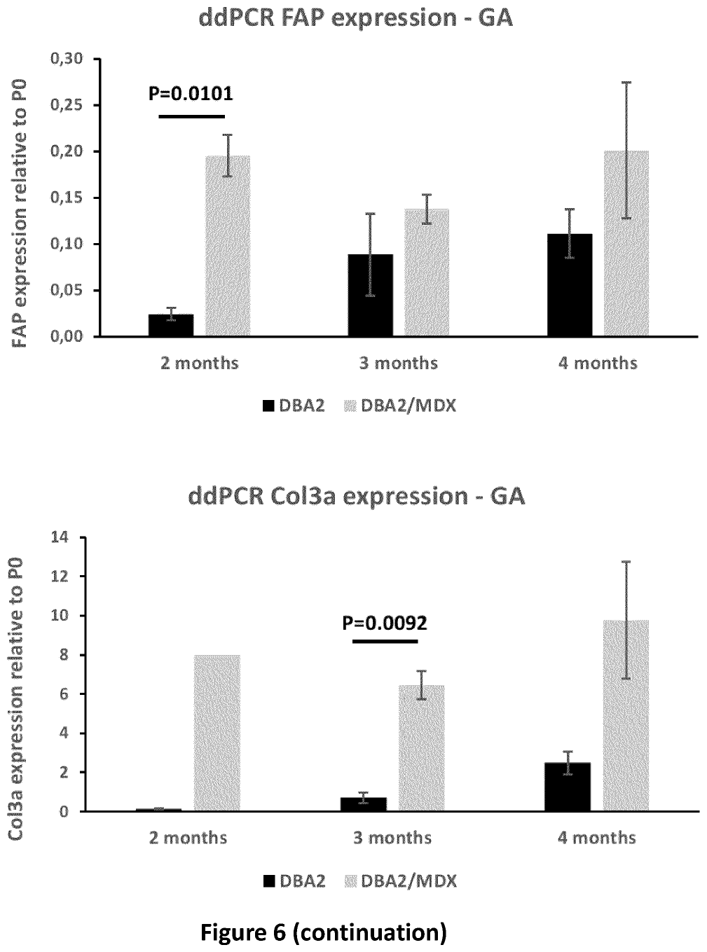

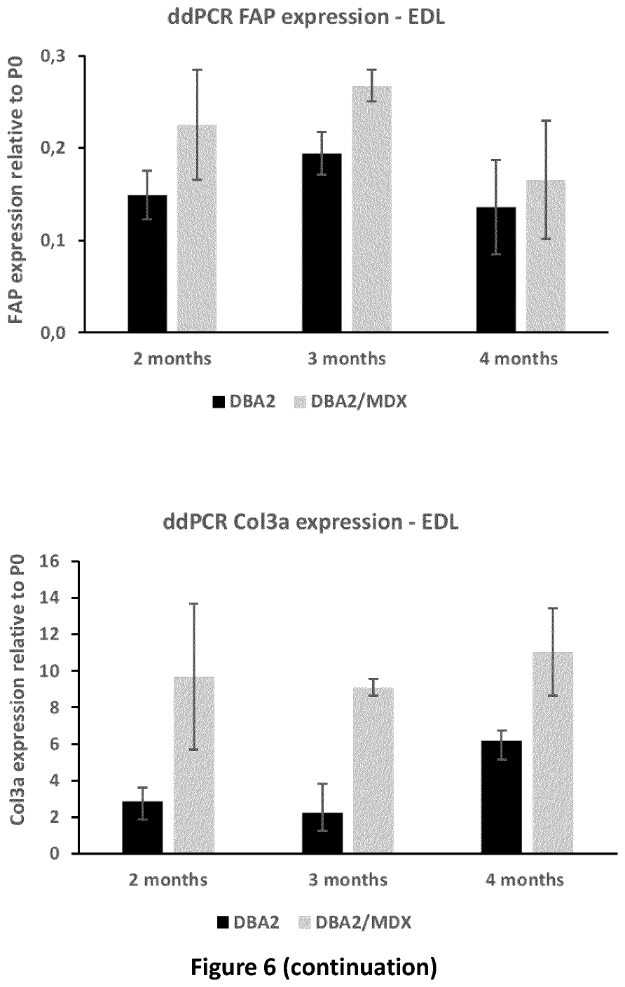

Biomarkers of fibrosis such as FAP expression as well as another marker of fibrosis, Collagen III, were quantified in skeletal muscles of interest (TA=tibialis anterior; GA=gastrocnemius; EDL: extensor digitorum longus) in the DBA2-MDX mouse model for Duchenne muscular dystrophy, over time and in the steady-state, using ddPCR (FIG. 6). Results show that independently of the age, these two makers of fibrosis are always overexpressed in skeletal muscles from DBA2-MDX compared to their age-match DBA2 controls. Significant differences are identified in the TA at 3 and 4 months, and in the GA at 2 or 3 months, depending on the biomarker. Similar trends are also seen in the EDL although it did not reach statistical significance. Lack of significance for some groups might be due to low mice number per group (n=3).

### 5. Effect of FAP-CAR-T Cells In Vivo on DBA2-MDX Associated Fibrosis

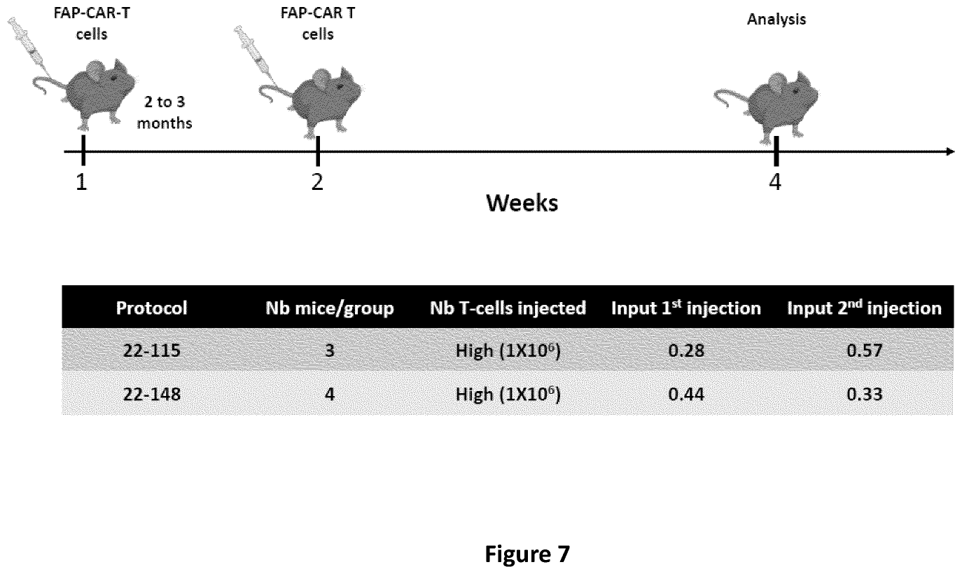

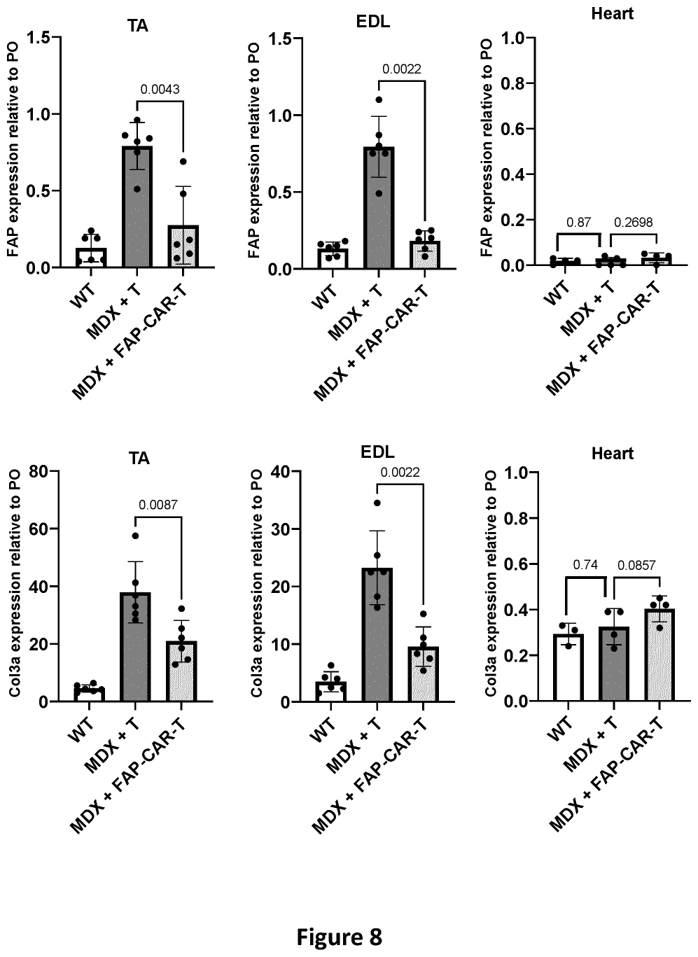

To test the efficacy of the FAP-CAR-T treatment on DBA2-MDX-associated fibrosis in vivo, two to three month-old DBA2-MDX mice were injected with two consecutive doses of FAP-CAR-T cells prepared and characterized as previously described (see schematic representation of the protocol in FIG. 7). In order to assess the effects of CAR-T cells on fibrosis, mRNA from skeletal muscles of control or treated mice were extracted and after reverse transcription, expression of FAP and Collagen III were determined by ddPCR (FIG. 8). Results show that FAP-CAR-T cells injected systemically are able to decrease biomarkers of fibrosis in skeletal muscles in DBA2-MDX mice. The reduction of biomarkers by FAP-CAR T cells is more pronounced in the TA and EDL where these two biomarkers are expressed at higher levels compared to the GA. Results show also that injection of FAP-CAR-T cells has no significant impact on the decrease biomarkers of fibrosis in heart in DBA2-MDX mice because FAP is not expressed (FIG. 8).

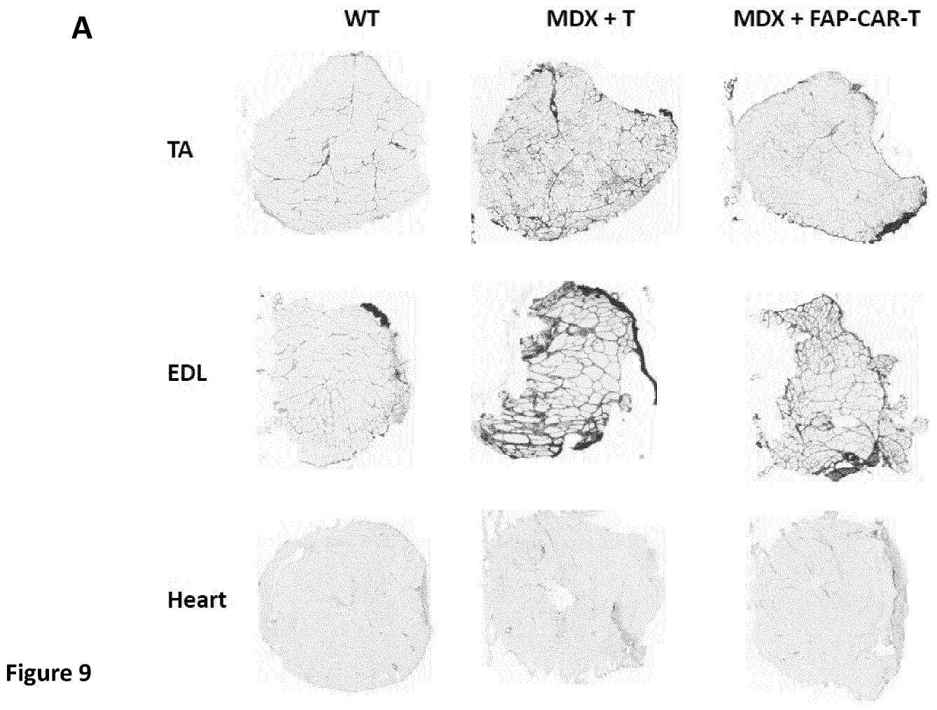

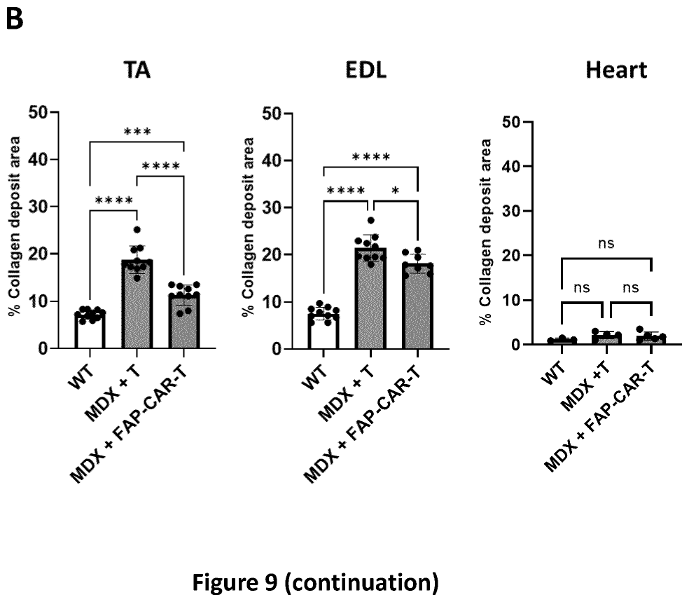

To confirm effect on fibrosis, histological sections of skeletal muscles from control and treated mice were stained with Sirius red in order to visualize the amount of collagen fibers present in the tissue. Results show a decrease in collagen in skeletal muscle from DBA-MDX treated with the FAP-CAR-T as well as an improvement in the structural organization of muscle fibers (FIGS. 9A and 9B).

### 6. Effect of FAP-CAR-T Cells Mediated Fibrosis Reduction on Gene Therapy in DMD Mouse Model

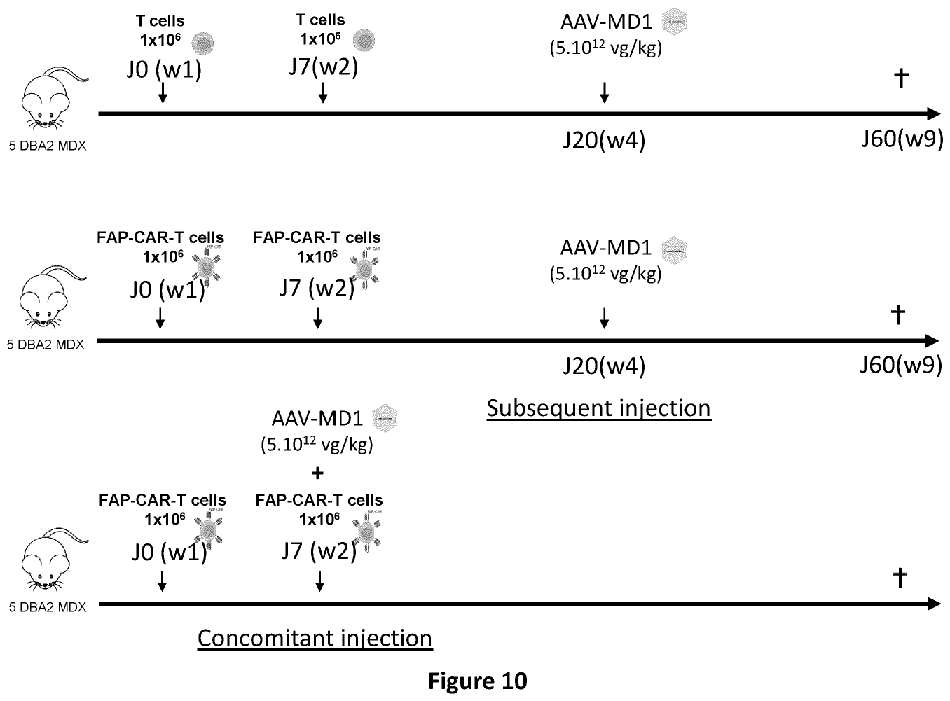

To evaluate the effect of the FAP-CAR-T mediated fibrosis reduction on gene therapy for Duchenne Muscular Dystrophy, two combinations of treatment are being tested: subsequent and concomitant injections of FAP-CAR-T and rAAV vector expressing microdystrophin. For subsequent gene therapy, two to three-month-old DBA2-MDX mice were injected with two consecutive doses of FAP-CAR-T cells, and after two weeks of treatment the mice were injected with one dose of rAAV9-microdystrophin (MD1) vector. For concomitant gene therapy, two to three-month-old DBA2-MDX mice were injected with one dose of FAP-CAR-T cells, and after two weeks of treatment the mice were injected concomitantly with a second dose of FAP-CAR-T cells and one dose of rAAV9-microdystrophin (MD1) vector (FIG. 10). Different tests were performed to assess the expression of microdystrophin mRNA and protein, to assess the quantity of AAV vectors copies in muscle and to evaluate the muscle integrity from control or treated mice.

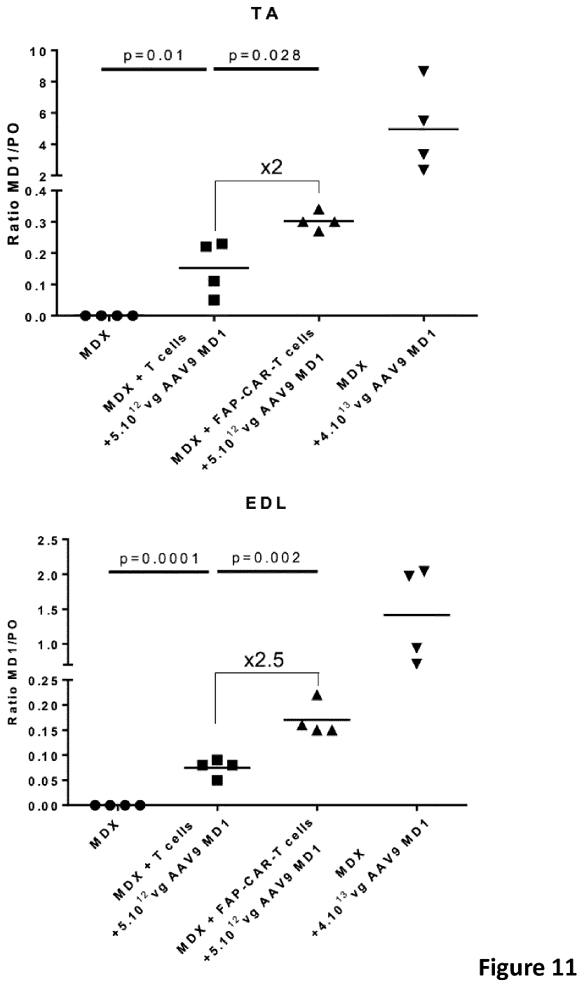

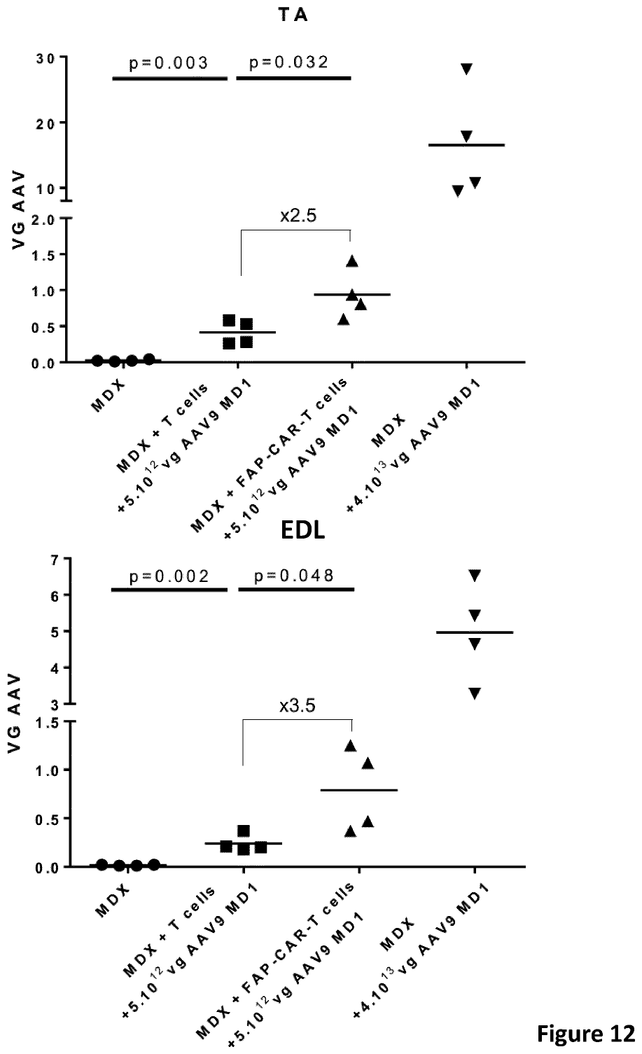

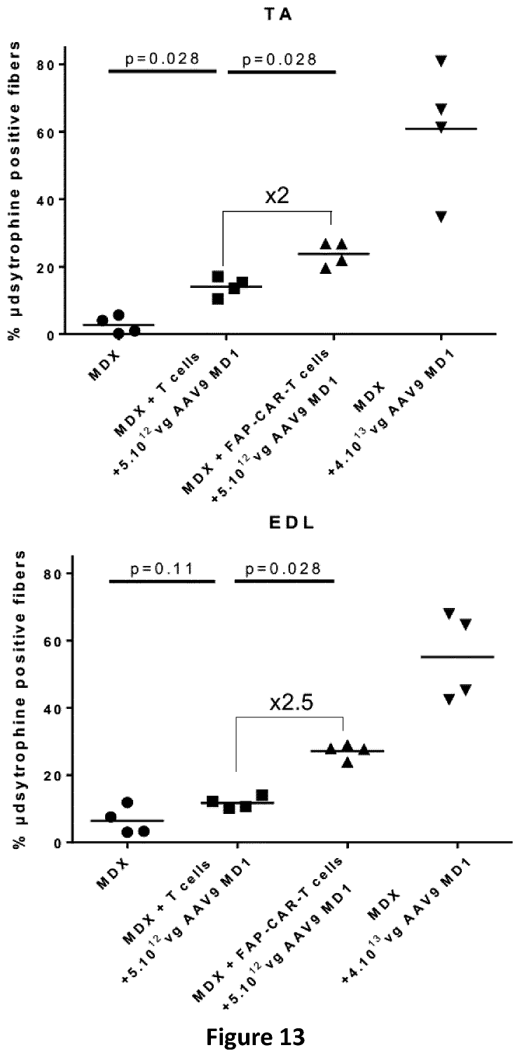

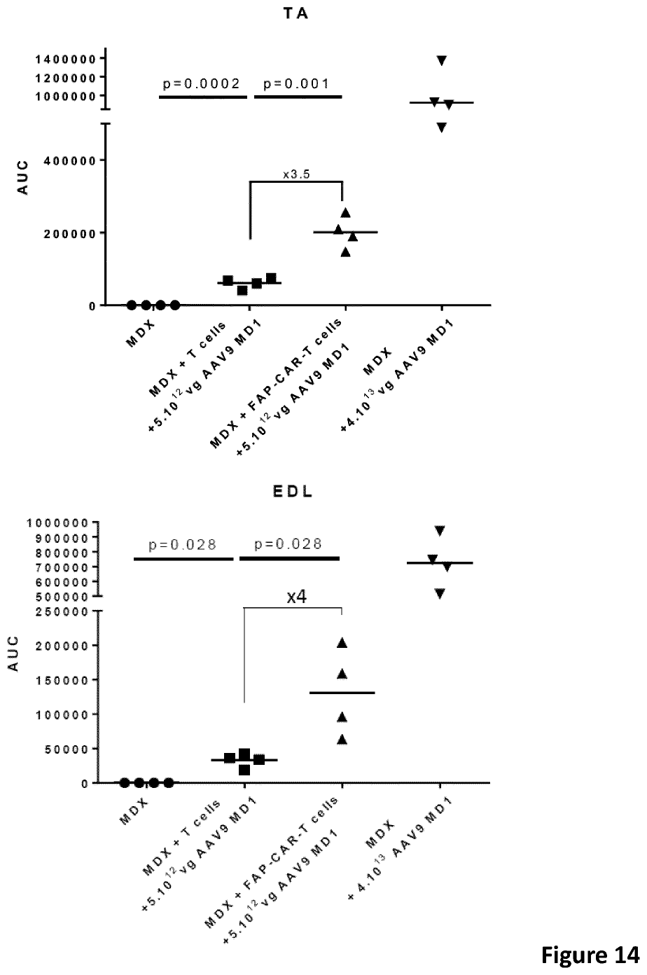

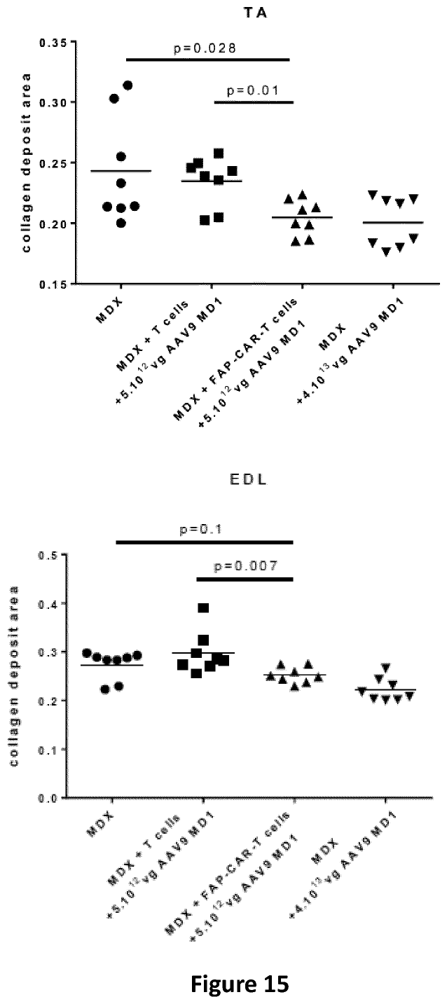

Results of subsequent gene therapy show that FAP-CAR-T cells mediatedfibrosis reduction systemically leads to highermicrodystrophin expression levels in muscle fibers in skeletal muscles in DBA2-MDX mice (FIGS. 11 and 14). There is an increase of AAV vectors copies in muscle and an increase of microdystrophin positive fibers (FIGS. 12 and 13). These effects seem to be more pronounced in the TA and the EDL where the the fibrosis is reduced. To confirm effect on muscle integrity, histological sections of skeletal muscles from control and treated mice were stained with Sirius red and hemotoxylin eosine in order to visualize the amount of collagen fibers present in the tissue. Results show a decrease in collagen in skeletal muscle from DBA-MDX treated with the FAP-CAR-T (FIG. 15) as well as an improvement in the structural organization of muscle fibers. Functional analyses of the skeletal muscle are being performed to confirm the efficacy of the combined therapy approach: FAP-CAR-T cells and AAV-MD1, to improve gene therapy for Duchenne muscular dystrophy.

For concomitant injections of FAP-CAR-T cells and AAV-MD1, it is expected that levels of dystrophin expression and reduction of collagen deposits are less important than in the case of subsequent injections of FAP-CAR-T cells and AAV-MD1.

1. A method for treating skeletal muscle fibrosis in muscular dystrophies comprising administering to a patient in need thereof a therapeutically effective amount of an immune cell engineered to express a chimeric antigen receptor (CAR) which specifically binds Fibroblast Activation Protein (FAP).

2. The method according to claim 1, wherein the CAR comprises: (i) an extracellular domain comprising at least one antigen-binding domain that specifically binds FAP, (ii) a transmembrane domain, and (iii) an intracellular domain comprising an intracellular signaling domain capable of activating an immune cell, and optionally comprising one or more co-stimulatory signaling domains.

3. The method according to claim 1, wherein the antigen-binding domain is a single-chain variable fragment (scFv) of a monoclonal antibody that specifically binds FAP.

4. The method according to claim 3, wherein the single-chain variable fragment (scFv) that binds FAP comprises a heavy chain variable domain comprising the amino acid sequence SEQ ID NO: 1 and a light chain variable domain comprising the amino acid sequence SEQ ID NO: 2.

5-15. (canceled)

16. The method according to claim 3 wherein the single-chain variable fragment (scFv) that binds FAP comprises the amino acid sequence SEQ ID NO: 8.

17. The method according to claim 2, wherein the extracellular domain further comprises a hinge domain from IgG4 heavy chain.

18. The method according to claim 2, wherein the transmembrane domain is from CD28.

19. The method according to claim 2, wherein the intracellular signaling domain is a CD3 zeta signaling domain.

20. The method according to claim 2, wherein the intracellular domain further comprises one or more co-stimulatory signaling domains from CD28 or 4-1BB.

21. The method according to claim 2, wherein the CAR comprises from its N- to C-terminus: a signal peptide from mouse Ig-kappa light chain, a scFv fragment from an anti-FAP monoclonal antibody, a modified hinge domain from human IgG4 heavy chain, a transmembrane domain from human CD28, a first co-stimulatory domain from human CD28, a second co-stimulatory domain from human 4-1BB and an intracellular signaling domain from human CD3 zeta chain.

22. The method according to claim 2 wherein the CAR comprises the amino acid sequence SEQ ID NO:16.

23. The method according to claim 1, wherein said immune cell is a T cell or NK cell.

24. The method according to claim 1 wherein said immune cell is a cytolytic T cell.

25. The method according to claim 1, wherein said immune cell is modified with an expression vector comprising a nucleic acid construct encoding the CAR chosen from a lipid nanoparticle packaging an RNA molecule and a lentiviral vector.

26. The method according to claim 25 wherein said expression vector is a self-inactivating or VSVG pseudotyped lentiviral vector.

27. The method according to claim 1, which reduces the expression level of at least one biomarker of fibrosis.

28. The method according to claim 1, wherein said immune cell is administered in combination with a vector for gene therapy of muscular dystrophies.

29. The method according to claim 1, wherein the muscular dystrophies are chosen from Dystrophynopathies, Limb-girdle muscular dystrophies and Congenital muscle dystrophies.

30. The method according to claim 1, wherein the muscular dystrophies is Duchenne muscular dystrophy.

31. The method of claim 28, wherein said immune cell is administered in combination with a reduced dose of vector for gene therapy compared to the use of the vector for gene therapy without the immune cell.
